SEQUENCE LISTING (13 sequences)

SEQ ID NO: 1 - DNA, 1797 bp, Homo sapiens
atgccttgtgttcaggcgcagtatgggtcctcgcctcaaggagccagccccgcttctcagagctacagttaccactcttcgggagaatacagctccgatttcttaactccagagtttgtcaagtttagcatggacctcaccaacactgaaatcactgccaccacttctctccccagcttcagtacctttatggacaactacagcacaggctacgacgtcaagccaccttgcttgtaccaaatgcccctgtccggacagcagtcctccattaaggtagaagacattcagatgcacaactaccagcaacacagccacctgcccccccagtctgaggagatgatgccgcactccgggtcggtttactacaagccctcctcgcccccgacgcccaccaccccgggcttccaggtgcagcacagccccatgtgggacgacccgggatctctccacaacttccaccagaactacgtggccactacgcacatgatcgagcagaggaaaacgccagtctcccgcctctccctcttctcctttaagcaatcgccccctggcaccccggtgtctagttgccagatgcgcttcgacgggcccctgcacgtccccatgaacccggagcccgccggcagccaccacgtggtggacgggcagaccttcgctgtgcccaaccccattcgcaagcccgcgtccatgggcttcccgggcctgcagatcggccacgcgtctcagctgctcgacacgcaggtgccctcaccgccgtcgcggggctccccctccaacgaggggctgtgcgctgtgtgtggggacaacgcggcctgccaacactacggcgtgcgcacctgtgagggctgcaaaggcttctttaagcgcacagtgcaaaaaaatgcaaaatacgtgtgtttagcaaataaaaactgcccagtggacaagcgtcgccggaatcgctgtcagtactgccgatttcagaagtgcctggctgttgggatggtcaaagaagtggttcgcacagacagtttaaaaggccggagaggtcgtttgccctcgaaaccgaagagcccacaggagccctctcccccttcgcccccggtgagtctgatcagtgccctcgtcagggcccatgtcgactccaacccggctatgaccagcctggactattccaggttccaggcgaaccctgactatcaaatgagtggagatgacacccagcatatccagcaattctatgatctcctgactggctccatggagatcatccggggctgggcagagaagatccctggcttcgcagacctgcccaaagccgaccaagacctgctttttgaatcagctttcttagaactggttgtccttcgattagcatacaggtccaacccagtggagggtaaactcatcttttgcaatggggtggtcttgcacaggttgcaatgcgttcgtggctttggggaatggattgattccattgttgaattctcctccaacttgcagaatatgaacatcgacatttctgccttctcctgcattgctgccctggctatggtcacagagagacacgggctcaaggaacccaagagagtggaagaactgcaaaacaagattgtaaattgtctcaaagaccacgtgactttcaacaatggggggttgaaccgccccaattatttgtccaaactgttggggaagctcccagaacttcgtaccctttgcacacaggggctacagcgcattttctacctgaaattggaagacttggtgccaccgccagcaataattgacaaacttttcctggacactttacctttctaa

SEQ ID NO: 2 - DNA, 27 bp, Artificial
  primer
gaagatctagagagacgcggagaactc

SEQ ID NO: 3 - DNA, 27 bp, Artificial
  primer
ccgctcgagttagaaaggtaaagtgtc

SEQ ID NO: 4 - DNA, 19 bp, Artificial
  primer
agtagggtcctcgcctcaa

SEQ ID NO: 5 - DNA, 24 bp, Artificial
  primer
attctcccgaagagtggtaactgt

SEQ ID NO: 6 - DNA, 21 bp, Artificial
  primer
tgccacgctgtactggttcac

SEQ ID NO: 7 - DNA, 19 bp, Artificial
  primer
gtcgaaggcccgaatctca

SEQ ID NO: 8 - DNA, 24 bp, Artificial
  primer
acagacttaacgggagcctttaga

SEQ ID NO: 9 - DNA, 23 bp, Artificial
  primer
gtgataagccctgaatcctgatg

SEQ ID NO: 10 - DNA, 23 bp, Artificial
  primer
catagacggcatcagagcatacc

SEQ ID NO: 11 - DNA, 19 bp, Artificial
  primer
ccgcgtcaatccaaacaga

SEQ ID NO: 12 - DNA, 20 bp, Artificial
  primer
tggtatcgtggaaggactca

SEQ ID NO: 13 - DNA, 20 bp, Artificial
  primer
agtgggtgtcgctgttgaag

(19) United States
(12) Patent Application Publication
(10) Pub. No.: US 2009/0081171 A1
(43) Pub. Date: Mar. 26, 2009

(54) Cell system for alleviating syndromes of Parkinson's disease in a mammal

(76) Inventors: Yu-Show Fu, Taipei (TW); Henrich Cheng, Taipei (TW)

Correspondence Address: PANITCH SCHWARZE BELISARIO & NADEL LLP, ONE COMMERCE SQUARE, 2005 MARKET STREET, SUITE 2200, PHILADELPHIA, PA, 19103, US

(21) Appl. No.: 11/891,935

(22) Filed: Aug. 13, 2007

Related U.S. Application Data
(60) Provisional application No. 60/822,213, filed on Aug. 11, 2006.

(51) Int. Cl. A61K 35/12

(52) U.S. Cl. 424/93.21; 424/93.7

(57) ABSTRACT

A cell system for treating neurodegenerative disorders in a mammal is provided. The cell system includes a population of neurons differentiated from umbilical mesenchymal stem cells for expressing at least one of tyrosine hydroxylase (TH), dopamine-β-hydroxylase (DBH), glutamate decarboxylase (GAD), aromatic L-amino acid decarboxylase (AADC) and dopaminergic transporter (DAT) in a cell culture. A method for treating neurodegenerative disorders in a mammal is also provided. The method comprises the steps of differentiating umbilical mesenchymal stem cells into a population of neurons that express at least one of TH, DBH, GAD, AADC and DAT in a cell culture, and transplanting the population of neurons into the brain of the mammal.

### CROSS-REFERENCE TO RELATED APPLICATION

This application claims the benefit of U.S. Provisional Application No. 60/822,213, filed Aug. 11, 2006, the entire disclosure of which is hereby incorporated by reference herein.

### BACKGROUND OF THE INVENTION

The present invention relates to a cell system and method for treating a neurodegenerative disorder in a mammal, more particularly to a cell system and method for alleviating syndromes of Parkinson's disease in the mammal.

Parkinson's disease is a neurodegenerative disorder characterized by the progressive loss of striatal dopaminergic function (Hornykiewicz et al., Pharmacol Rev 18:925-964 (1996); Bernheimer et al., J Neurol Sci 20:415-455 (1973); Nagatsu et al., Adv Neurol 40: 467-473 (1984); Agid et al., Biochemistry of neurotransmitter in Parkinson's disease, 2^{nd} edition, Butterworths, London (1987); Kish et al., N Engl J Med 318: 876-880 (1988); Damier et al., Brain 122: 1437-1448 (1999)). Patients initially respond to treatment with dopaminergic enhancing medications such as levodopa (L-DOPA) (Cotzias et al., N Engl J Med 276:374-379 (1967)). However, the effectiveness of such treatments gradually diminishes because the conversion to dopamine within the brain is increasingly disrupted by the progressive degeneration of the dopaminergic terminals. As a result, after approximately ten years of dopamine-replacement treatment, most patients with Parkinson's disease still suffer from disability that cannot be satisfactorily controlled (Olanow et al., Annu Rev Neurosci 22:123-144 (1999)).

An alternative approach for the restoration of the damaged dopaminergic system involves transplanting cells (or tissues) that synthesize catecholamines (Backlund et al., J Neurosurg 62: 169-173 (1985); Madrazo et al., N Engl J Med 318: 51 (1988); Lindvall O., J Neuro Neurosurg Psychiatry Suppl: 39-54 (1989); Date et al., J Neurosurg 84: 685-689 (1996); Deacon et al., Nat Med 3: 350-353 (1997)). There is evidence both from animal studies and clinical investigations showing that fetal dopamine neurons can produce symptomatic relief (Mahowald et al., Science 235: 1307-1308; Spencer et al., N Eng J Med 327: 1541-1548 (1992); Freed et al., N Eng J Med 327: 1549-1555 (1992); Kordower et al., N Eng J Med 332: 1118-1124 (1995); Olanow et al., Trends Neurosci 19: 102-109 (1996); Kordower et al., Mov. Disord 13: 383-393 (1998); Hauser et al., Arch Neurol 56: 179-87 (1999); Lindvall O., Mov. Disord 14: 201-205 (1999); Piccini et al., Nat Neurosci 2: 1137-1140 (1999); Freed et al., N Eng J Med 344: 710-719 (2001): Clarkson E. D., Drugs Aging 18: 773-785 (2001); Mendez et al., J Neurosurg 96: 589-596 (2002); Ben-Hur et al., Stem Cells 22: 1246-1255 (2004)). However, technical and ethical difficulties in obtaining sufficient and appropriate graft tissues have limited the application of this therapy (Greely et al., N Engl J Med 320:1093-1096 (1989)).

### BRIEF SUMMARY OF THE INVENTION

One aspect of the invention relates to a cell system for treating neurodegenerative disorders in a mammal. The cell system comprises a population of neurons differentiated from umbilical mesenchymal stem cells for expressing at least one of tyrosine hydroxylase (TH), dopamine-β-hydroxylase (DBH), glutamate decarboxylase (GAD), aromatic L-amino acid decarboxylase (AADC) and dopaminergic transporter (DAT) in a cell culture.

Another aspect of the invention relates to a cell system for alleviating at least one syndrome of Parkinson's disease in a mammal. The cell system comprises a population of neurons differentiated from human umbilical cord mesenchymal stem cells for expressing TH in a cell culture.

A further aspect of the invention relates to a method for treating at least one neurodegenerative disorder in a mammal. A population of umbilical mesenchymal stem cells is differentiated into neurons that express at least one of TH, DBH, GAD, AADC and DAT in a cell culture. The neurons are then transplanted into the brain of the mammal.

One other aspect of the invention relates to a method for alleviating at least one symptom of Parkinson's disease in a mammal. A population of umbilical mesenchymal stem cells is isolated from Wharton's jelly of a human umbilical cord. Next, the population of umbilical mesenchymal stem cells is differentiated into neurons that express TH in a cell culture. The neurons are then transplanted into the brain of the mammal.

### BRIEF DESCRIPTION OF THE SEVERAL VIEWS OF THE DRAWINGS

The patent or application file contains at least one drawing executed in color. Copies of this patent or patent application publication with color drawings will be provided by the Office upon request and payment of the necessary fee.

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1A is a histogram showing the percentages of TH positive cells after incubation with neuronal conditioned medium (NCM), sonic hedgehog protein (SHH) and fibroblast growth factor 8 (FGF8);

FIG. 1B is a histogram showing the dopamine concentration in culture medium after human umbilical mesenchymal stem cells (HUMSCs) are treated with NCM, SHH and FGF8;

FIG. 2 is a series of images of rat brain showing the extent of HUMSCs migration after implantation in the striatum of the rat at the bregma level;

FIG. 3 is a line graph showing the rotational behavior of Parkinsonian rats in response to amphetamine after transplanting the HUMSCs treated with NCM, SHH and FGF8;

FIG. 4 is a flow chart showing the HUMSCs differentiated into dopamine-neuron after the HUMSCs were transfected with pCMV-HA or pCMV-HA-Nurr1 vector, followed by treating with NCM, SHH and FGF8 in different manners;

FIG. 5A is a map showing the structure of Nurr1 mRNA, and FIG. 5B is an image showing the size of Nurr1 cDNA fragment;

FIG. 6A is a map showing the construction of pCMV-HA-Nurr1 vector, and FIG. 6B is an image showing the size of a whole pCMV-HA-Nurr1 vector, empty pCMV-HA vector fragment and Nurr1 cDNA fragment only;

FIG. 7A is an image of Nurr1 gene expression of HUMSCs only and HUMSCs transfected with Nurr1 gene measured by real-time PCR, and FIG. 7B is a histogram showing the Nurr1/GADPH ratio in the HUMSCs 24 hours after the Nurr1 gene is transfected;

FIGS. 8A-8C are histograms showing TH/GADPH, AADC/GADPH and DAT/GADPH ratios, respectively, after the HUMSCs are transfected with pCMV-HA or pCMV-HA-Nurr1 vector and treated with FGF8 and SHH for 3 days and NCM for 6 days;

FIGS. 9A and 9B are images showing the morphology change of the HUMSCs after the HUMSCs were transfected with pCMV-HA-Nurr1 vector and treated with FGF8 and SHH for 3 days and NCM for 6 days;

FIGS. 10A, 10B and 10C are color images showing the TH positive neurons, which were differentiated from the HUMSCs after different treatments;

FIG. 11 is an image showing the cellular expression of TH from the HUMSCs as the HUMSCs were treated with different manners;

FIG. 12 is a histogram showing the percentage of TH⁺ neurons observed after the HUMSCs are transfected with pCMV-HA or pCMV-HA-Nurr1 vector and treated with at least one of NCM, SHH and FGF8 for various periods;

FIG. 13 is a histogram showing the dopamine concentration in culture medium after HUMSCs are treated with at least one of NCM, SHH and FGF8 for various periods;

FIGS. 14A and 14B are HPLC spectra showing the presence of dopamine, serotonin and their metabolites in the culture medium when the HUMSCs are transfected with pCMV-HA-Nurr1 vector (FIG. 14A) and with pCMV-HA vector (FIG. 14B), and each respectively treated with FGF8 and SHH for 3 days and NCM for 6 days;

FIG. 15 is a histogram showing the dopamine level in cells after the HUMSCs are transfected with pCMV-HA or pCMV-HA-Nurr1 vector and treated with FGF8 and SHH for 3 days and NCM for 6 days;

FIGS. 16A and 16B are color images showing the cell with bis-benzamide at the implication site;

FIGS. 17A and 17B are color images showing the presence of ¹⁸F-dopamine at the injured right striatum in the rat of the control group (FIG. 17A) and experimental group (FIG. 17B); and

FIG. 18 is a color line graph showing the rotational behavior of the Parkinsonian rats in response to amphetamine after transplanting the HUMSCs transfected with pCMV-HA or pCMV-HA-Nurr1 vector and treated with NCM, SHH and FGF8.

### DETAILED DESCRIPTION OF THE INVENTION

The present invention relates to a cell system for treating neurodegenerative disorders in a mammal, preferably a human. A population of umbilical mesenchymal stem cells is isolated from an umbilical cord of a mammal, preferably a human. The population of umbilical mesenchymal stem cells is differentiated into neurons that express at least one of tyrosine hydroxylase (TH), dopamine-β-hydroxylase (DBH), glutamate decarboxylase (GAD), aromatic L-amino acid decarboxylase (AADC) and dopaminergic transporter (DAT) in a cell culture. The cell culture containing the umbilical mesenchymal stem cells and differentiated cells is then transplanted into the brain of the mammal. As used herein, the terms “transplant,” “implant” and “graft” may be used interchangeably. As used herein, the singular forms “a,” “an” and “the” include plural referents unless the context clearly dictates otherwise.

TH, DBH, GAD, AADC and DAT are several key enzymes and proteins in the synthesis and regulation of catecholamine neurotransmitters (such as norepinephrine, epinephrine and dopamine). DAT is a transporter for the uptake of dopamine from the synapse. Catecholamine level irregularities have been uncovered in the neurodegenerative disorders such as Parkinson's disease, Huntington's disease, Alzheimer's disease, schizophrenia, epilepsy, dystonia and so on. Accordingly, the neurodegenerative disorders associated with irregular catecholamine levels may be treated by transplanting the differentiated cells that express at least one of TH, DBH, GAD, AADC and DAT into the brain of the mammal at an area of degeneration to regulate the catecholamine levels.

In accordance with one embodiment of the invention, a mesenchymal tissue or Wharton's jelly is dissociated from a human umbilical cord. The population of umbilical mesenchymal stem cells is then isolated from the mesenchymal tissue of the human umbilical cord. The umbilical mesenchymal stem cells are cultured with a composition comprising a mammalian hedgehog and mammalian fibroblast growth factor (FGF) in a culture medium, so that the umbilical mesenchymal stem cells are differentiated into neurons expressing at least one of TH, DBH and GAD. The culture medium may include a neuronal conditioned medium (NCM) produced by culturing neuronal cells of the mammal in a basal medium, such as Dulbecco's Modified Eagles' Medium (DMEM) supplemented with 10% fetal bovine serum (FBS) (10% FBS-DMEM), 10% FBS-DMEM without any prior culturing in the presence of the neurons, and a combination of both the NCM and 10% FBS-DMEM. However, it should be noted that other types of the basal media suitable for maintaining growth of the umbilical mesenchymal stem cells may also be used and cultured with the neuronal cells to provide the culture medium.

In accordance with another embodiment of the invention, a population of umbilical mesenchymal stem cells is isolated from the mesenchymal tissue of the human umbilical cord. A transgene, such as the Nurr1 gene of SEQ ID NO:1 is then introduced into the population of umbilical mesenchymal stem cells. In some embodiments of the invention, the population of umbilical mesenchymal stem cells is transfected with an expression system comprising at least the Nurr1 gene of SEQ ID NO:1. It is understood that the expression system further includes other corresponding regulatory elements well known to one skilled in the art for regulating expression of the transgene. Therefore, the details of other regulatory elements in the expression system are omitted. It is also noted that the method by which the transgene is introduced into the population of umbilical stem cells is not limited to transfection. Other methods for introducing the transgene into the mesenchymal stem cells are well known to one skilled in the art, and are thus encompassed by the scope of the present invention. In accordance with yet another embodiment of the invention, the population of umbilical mesenchymal stem cells may be transfected with an expression system comprising at least the Nurr1 gene of SEQ ID NO:1, followed by culturing with a composition comprising a mammalian hedgehog and mammalian FGF in a culture medium, so that the umbilical mesenchymal stem cells are differentiated into neurons that express at least one of TH, AADC and DAT.

The present invention also includes a method for treating neurodegenerative disorders in a mammal, preferably a human. The population of umbilical mesenchymal stem cells is differentiated into neurons that express at least one of TH, DBH, GAD, AADC and DAT in a cell culture. The cell culture containing umbilical mesenchymal stem cells and differentiated cells is then transplanted into the brain of the mammal.

In accordance with another embodiment of the invention, the population of umbilical mesenchymal stem cells isolated from an umbilical cord of a mammal is cultured with a composition comprising a mammalian hedgehog, such as a murine N-terminal fragment of sonic hedgehog protein (SHH), and a mammalian FGF, such as a murine FGF8 isoform b in a culture medium. However, the composition is not limited to include only the murine N-terminal fragment of SHH and murine FGF8 isoform b. Other mammalian hedgehogs, FGFs and their isoforms may be included in the composition as long as they can promote differentiation of the umbilical mesenchymal stem cells into neurons that express at least one of TH, DBH and GAD.

In some embodiments of the invention, the population of umbilical mesenchymal stem cells is cultured with the neuronal conditioned medium for expanding the population of umbilical mesenchymal stem cells, followed by culturing with a composition comprising the murine N-terminal fragment of SHH, and the murine FGF8 isoform b in the NCM for a period of 3, 6, 9 or 12 days. As a result, the umbilical mesenchymal stem cells are differentiated into a population of neurons that express at least one of TH, DBH and GAD. In other words, the umbilical mesenchymal stem cells are differentiated into subpopulations of dopaminergic, norepinephrine and GABAergic neurons.

In accordance with other embodiments of the invention, the population of umbilical mesenchymal stem cells is cultured with the neuronal conditioned medium for expanding the population of umbilical mesenchymal stem cells, followed by culturing with a composition comprising the murine N-terminal fragment of SHH, and the murine FGF8 isoform b in 10% FBS-DMEM for a period of 3, 6, 9 or 12 days. As a result, the umbilical mesenchymal stem cells are differentiated into subpopulations of dopaminergic, norepinephrine and GABAergic neurons.

The population of umbilical mesenchymal stem cells is also genetically modified prior to transplantation. For example, a transgene such as human Nurr1 of SEQ ID NO:1 is introduced into the umbilical mesenchymal stem cells. Methods of introducing the transgene into the cells are known in the art, and include transfection with an expression vector and infection with viral vectors, such as retrovirus, herpes virus, adenovirus, and adeno-associated virus vectors. The cells transfected with the expression vector comprising the Nurr1 gene of SEQ ID NO:1 may be further cultured with the NCM, followed by culturing with a composition comprising the murine N-terminal fragment of SHH, and the murine FGF8 isoform b in the NCM, so that the umbilical mesenchymal stem cells are differentiated into neurons where TH, AADC and DAT may be constitutively expressed to regulate the catecholamine levels in the brain of the mammal.

Thereafter, the neurons are transplanted into the brain of the mammal to reconstitute damaged neural circuits, and/or replace lost neurons and neurotransmitter systems in the treatment of the neurodegenerative disorders including but not limited to Parkinson's disease, Huntington's disease, Alzheimer's disease, schizophrenia, epilepsy and dystonia. It would be understood by one skilled in the pertinent art in view of the present disclosure that the cell system is also applicable to the treatment of other neurodegenerative disorders associated with catecholamine imbalance within the brain.

The present invention also includes a cell system for alleviating syndromes of Parkinson's disease in a mammal, preferably a human. The cell system comprises a population of human umbilical mesenchymal stem cells (HUMSCs) differentiated into neurons that express TH in a cell culture. In accordance with one embodiment of the invention, the population of HUMSCs is cultured with a composition comprising the murine N-terminal fragment of SHH and the murine FGF8 isoform b in the culture medium. As a result, the HUMSCs are differentiated into dopaminergic neurons that express TH. Next, the dopaminergic neurons are transplanted into the brain of the mammal at an area of neurodegeneration including but not limited to substantia nigra, striatum, basal ganglia and other brain regions where the dopaminergic neurons reside or originate, resulting in reconstitution of damaged neural circuits, and/or replacement of lost neurons and neurotransmitter systems.

The syndromes of Parkinson's disease usually include but are not limited to tremor or trembling of the limbs, jaw and face; stiffness or rigidity of the limbs and trunk; slowness of movement; and postural instability or impaired balance or coordination. Furthermore, the syndromes of Parkinson's disease may also be induced with a drug such as 6-hydroxydopamine HCl (6-OHDA) in an animal model. As a result, the animal model may exhibit lesion-induced amphetamine evoked rotation as one syndrome of Parkinson's disease in the animal model.

The present invention also relates to a method for alleviating symptoms of Parkinson's disease in a mammal, preferably a human. A population of HUMSCs is isolated from Wharton's jelly of a human umbilical cord. The HUMSCs are differentiated into dopaminergic neurons that express TH. In accordance with one embodiment of the invention, the HUMSCs are cultured with a composition comprising murine N-terminal fragment of SHH and the murine FGF8 isoform b in a culture medium, so that the HUMSCs are differentiated into neurons that express TH. In accordance with another embodiment of the invention, the HUMSCs may be transfected with an expression system comprising at least the Nurr1 gene of SEQ ID NO:1, followed by culturing with a composition comprising murine N-terminal fragment of SHH and the murine FGF8 isoform b in the NCM, so that the HUMSCs are differentiated into neurons that express TH or TH⁺ neurons. Next, the TH⁺ neurons are transplanted into the brain, preferably into the striatum of the mammal to alleviate syndromes of the Parkinson's disease that occur as a result of loss of dopamine producing cells.

The invention will now be described in further detail with reference to the following specific, non-limiting examples. These examples should in no way be construed as limiting the scope of the invention, as defined by the appended claims. As used herein, in the examples and elsewhere, all percentages are percents by weight (w/w) of the indicated component or ingredient based on the whole composition or a larger component, as appropriate, unless otherwise indicated.

### EXAMPLE 1

### Transplanting HUMSCs Treated with SHH, FGF8 and NCM into the Brain of Parkinsonian Rat

Isolation and proliferation of HUMSCs

Human umbilical cords were obtained front an obstetrical clinic with the donors' consent. The umbilical cords were collected by aspectic manipulation and stored in Hank's Balanced Salt Solution (HBSS) (Biochrom, Berlin, Germany) below 4° C. for no more than 24 hours. The umbilical cord was disinfected in 75% ethanol for 30 s. The disinfected umbilical cord was placed in Ca²⁺/Mg²⁺ free buffer (CMF) in a germ free laminar flow, cut lengthwise with an autoclaved tool, and from which the blood vessels and the mesenchymal tissue in Wharton's jelly were removed. The mesenchymal tissue was then diced into cubes of about 0.5 cm³ and centrifuged at 250 g for 5 min. The mesenchymal tissue was treated with collagenase at 37° C. for 14 to 18 h, washed with serum-free DMEM (Gibco, BRL, USA), and further digested with 2.5% trypsin (Gibco, BRL, USA) at 37° C. for 30 min. FBS (HyClone Laboratory, Logan, Utah) was then added to the mesenchymal tissue to neutralize the excess trypsin. A population of umbilical mesenchymal stem cells dissociated from the mesenchymal tissue was further dispersed by treatment with 10% FBS-DMEM and counted under the microscope using a hemocytometer. The population of umbilical mesenchymal stem cells was either cultured for cell proliferation or stored in liquid nitrogen for later use.

Transformation of HUMSCs into TH⁺ Neurons

The neurons used for preparing the neuronal conditioned medium were obtained from the brain of a Sprague-Dawley rat of 7 days old (The Animal Center of National Yang Ming University, Taipei, Taiwan, R.O.C.). The rats were anaesthetized by intraperitoneal injection of 0.3 ml 10% chloride hydrate. After 3 to 4 minutes, the whole body of total anesthetized rat was disinfected with 75% ethanol. In a germ-free laminar flow, the brain of the rat was removed and placed in CMF (Gibco, BRL, USA), and centrifuged at 900 rpm for 5 minutes. Following removal of the supernatant fraction, 10% FBS-DMEM was added to the precipitate (brain tissue). The brain tissue suspension was triturated 15 times for dispersal into single cells. The cells were suspended in 10% FBS-DMEM and incubated at 37° C. in 5% CO₂ and 95% O₂. AraC (Sigma, St. Louis, Mo.) was added to the culture to a final concentration of 2 μM on the next day. On the 5th day of culture, the NCM was collected for culturing umbilical mesenchymal cells. The HUMSCs were cultured in the NCM and fresh NCM was replaced every other day.

In vitro differentiation of HUMSCs into TH⁺ neurons was carried out as previously described with modifications (Lee et al., Nat biotechnol 218:675-679 (2000)). At the cell expansion stage, a population of undifferentiated HUMSCS were dissociated into single cells and cultured in 10% FBS-DMEM for 3 to 6 days for expansion. Next, HUMSCs were cultured in NCM for 6 to 9 days, which was replaced every other day to induce neuron-like differentiation. Thereafter, the cells were then supplemented with NCM or 10% FBS-DMEM in the presence of the murine N-terminal fragment of sonic hedgehog protein (SHH, 500 ng/ml, R&D 461-SH) and murine FGF8 isoform b (FGF8, 100 ng/ml, R&D 423-F8) for 3, 6, 9, or 12 days, to allow differentiation of the cells into dopaminergic neurons expressing TH. A group of HUMSCs cultured in NCM or DMEM alone for 6 days was also provided as a control group to compare with those cultured with NCM, SHH and FGF8.

### Immunocytochemistry for TH

HUMSCs and brain sections of the rats were fixed with 4% paraformaldehyde in 0.1 M phosphate buffer for 20 min., and washed with 0.1 M phosphate buffer. The cells and brain sections were treated with a blocking solution (0.05% Triton X-100, 5% normal goat serum and 3% bovine serum albumin) for 30 min. in order to prevent nonspecific antibody-antigen binding. The cells and brain sections were then reacted with mouse anti-TH antibodies (Chemicon, Temecula, Calif.) at 4° C. for 18 h., and washed with 0.1 M PBS to remove any non-specific bindings. Next, cells and brain sections binding to the primary antibodies were reacted with secondary antibodies at room temperature for 1 h., and washed again with 0.1 M PBS to remove any non-specific bindings. The cell and brain sections binding to both primary and secondary antibodies were further reacted with ABC complex (ABC KIT, Vectorlabs PK-4000) at room temperature for 1 h., and washed with 0.1 M PBS to remove any non-specific bindings. The cells and brain sections binding to ABC complex were developed with 3,3′-diaminobenzidine (DAB) (5 mg DAB, 3.5 μl of 30% H₂O₂ in 10 ml of 50 mM Tris Buffer).

### Double Staining of Anti-Human-Specific Nuclear Antigen and Anti-TH or Anti-DBH or Anti-GAD

HUMSCs were double stained for human-specific nuclear antigen (Zhang et al., Nature Bio. 19:1129-1133 (2001)) and TH, DBH or GAD for assessing differentiation of HUMSCs into subpopulations of dopaminergic, norepinephrine or GABAergic neurons.

The HUMSCs and brain sections were fixed with 4% paraformaldehyde in 0.1 M phosphate buffer for 20 min. and then washed with 0.1 M phosphate buffer. The HUMSCs and brain sections were then treated with a blocking solution for 30 min. in order to prevent nonspecific antibody-antigen binding. The cells or brain sections were then reacted with mouse anti-human-specific nuclear antigen antibodies (Chemicon, Temecula, Calif.); mouse anti-TH antibodies (Chemicon, Temecula, Calif.); mouse anti-DBH antibodies (Chemicon, Temecula, Calif.); rabbit anti-GAD antibodies (Chemicon, Temecula, Calif.) at 4° C. for 18 h., washed with 0.1 M PBS, reacted with fluorescein-conjugated goat anti-mouse-IgG antibodies (Chemicon, Temecula, Calif.); Rhodamine-conjugated-goat anti-mouse-IgG (Chemicon, Temecula, Calif.); Rhodamine-conjugated goat anti-rabbit-IgG (Chemicon, Temecula, Calif.) at room temperature for 1 h. The cells or brain sections labeled with the antibodies were then observed under a fluorescence microscope.

### Western Blotting for TH

Cell membranes were prepared from HUMSCs cultured in NCM, SHH and FGF8 for varying periods. Following resolution on 20% SDS-PAGE, the cell proteins were blotted onto PVDF membranes which were then washed with Tris-buffered saline (TBS) with 0.9% NaCl (pH 7.3), immersed in the blocking solution (0.05% Triton X-100, 5% normal goat serum and 3% bovine serum albumin) for 60 min., washed with TBS again, and reacted with mouse anti-TH antibodies (Chemicon, Temecula, Calif.) at 4° C. for 12 to 18 h. After the reaction was completed, the PVDF membranes were washed with Triton Tris Buffer (0.05% Triton and 0.9% NaCl in 50 mM Tris-HCl, pH 7.3), immersed in the blocking solution for 60 min., and then reacted with secondary antibodies at room temperature for 1 h. The PVDF membranes containing the reaction products were washed with Tween-20 Tris-buffered saline (TTBS), reacted with Avidin-Biotin complex (ABC) (Vector Laboratories, Burlingame, Calif.) at room temperature for 1 h., washed again with TTBS, and finally developed with diaminobenzidine (DAB).

### High-Performance Liquid Chromatography (HPLC) Analysis of Dopamine Concentration

The culture medium was acidified with 0.1 N perchloric acid and centrifuged at 10,000 g for 10 min. The supernatant was immediately frozen in liquid nitrogen and stored at −70° C. until analysis. An HPLC with an electrochemical (EC) detection procedure was used to quantify dopamine content in the supernatant (Chiueh et al., J. Pharmacol. Exp. Ther. 225: 529-533 (1983)). The electrochemical procedure included applying an oxidizing potential of 0.75 volt. The mobile phase contained 2.1 g heptanesulfonic acid, 0.1 g ethylenediamine-tetraacetic acid, 3 ml phosphoric acid, 3.5 ml triethylamine and 130 ml acetonitrile in 850 ml deionized water. Dopamine was separated in a C-18 column (4.6 mm×150 mm). The retention time for dopamine was approximately 8 min.

### Preparation of Parkinsonian Animals

Adult Sprague-Dawley rats (250 to 300 g) were used in this study. The rats were anesthetized by administering chloride hydrate (400 mg/kg) intraperitoneally and placed in a stereotaxic frame. The dopamine-innervated striatum were unilaterally lesioned by injecting 6-hydroxydopamine HCl (6-OHDA) into the median forebrain bundle AP: −4.3 mm, RIL: +1.6 mm, H: −8.2 mm and AP: −4.0 mm, RIL: +1.8 mm, H: −8.0 mm (Nikkhah et al., Neuroscience 56(1): 33-43 (1993); Olsson et al., J. Neuroscience 15(5 Pt 2): 3863-3875 (1995)). Coordinates were set according to the atlas of Paxinos and Watson (Paxinos et al., The rat brain in stereotaxic coordinates, 2^{nd} edition (1986)). Each rat was given 30 μg of 6-OHDA dissolved in 5 μl of physiological saline containing 0.02% ascorbic acid. The rats were placed in individual plastic hemispherical bowls and allowed to habituate for 10 min. before being injected with a subcutaneous dose of Amphetamine (5 mg/kg). Amphetamine-induced rotational behavior was assessed at 4, 8, 12, 16 and 20 weeks after 6-OHDA injection. Left and right full-body turns were counted. The number of amphetamine-induced net rotation over a period of 60 min., starting 30 min. after injection, was recorded. Animals which displayed greater than 360 turns/hr ipsilaterally toward the lesioned side after a single dose of amphetamine were selected as Parkinsonian rats for cell grafting (Arbuthnott et al., Brain Research 27(2): 406-413 (1971); Ungerstedt et al., Brain Research 24(3): 485-493 (1970); Ungerstedt et al., Acta Physiologica Scandinavica 367: 49-68 (1971)).

### Transplantation of HUMSC-Derived TH⁺ Neurons

The HUMSCs subjected to in vitro differentiation were treated with 1 ug/ml bis-benzimide (Sigma B2883) for 24 h in order to label the cells. The cells were trypsinized at 37° C. for 5 min. with 0.25% trypsin and the dissociated cells were resuspended in PBS. A total of 1×10⁵ cells in a 10-μl suspension were transplanted into the striatum of each rat (anterior 1.0 mm, lateral 3.0 mm, ventral −6.0 mm), based on positioning from the bregma and skull surface. The needle stayed in place for a period of 10 min. before being withdrawn to allow settling of the cells. None of the rat hosts received any immunosuppression medications.

### Behavior Test

The rats were divided into three groups, with each group containing 12 rats. One month after 6-OHDA injection, each of the rats in group 1 (control group) was injected with phosphate-buffered saline (PBS) into their dopamine-denervated striata. Each of the rats in group 2 (NCM group) was injected with a suspension of 1×10⁵ graft cells that had been cultured in the NCM only, and each of the rats in group 3 (NCM+SHH+FGF8) was injected with a suspension of 1×10⁵ graft cells cultured with SHH and FGF8 in the NCM. The effects of stem cell transplantation were examined in 6-OHDA-lesioned animals by quantification of rotations in response to amphetamine. Rotational scores were examined at 1, 2, 3 and 4 months post-transplantation. All behavioral tests were performed in a closed room to avoid any environmental disturbance and assessed by an independent observer blinded to the treatments.

### Histological Examination of Grafted Brain Cryosections

For tracking of the transplanted cells, the cell membrane penetrating and DNA-binding fluorescence probe bis-benzimide was used. Twenty weeks after transplantation, the grafted rats were anesthetized terminally by administering intraperitoneally with an overdose of pentobarbital. The brains were removed, post-fixed in 4% paraformaldehyde in PBS at 4° C. for 24 hours, and sectioned into specimens. Next, the specimens were equilibrated in 10% sucrose in PBS at 4° C. for 4 hours, followed by equilibrating in 15% sucrose in PBS at 4° C. for 4 hours, and finally in 20% sucrose in PBS at 4° C. overnight. The specimens were then embedded in optimum cutting temperature (OCT) compound and frozen in liquid nitrogen. Frozen sections were cut into serial 30 μm thick slices using a cryostat. The tissues were stained with the fluorescent stain bis-benzimide and visualized under a fluorescence microscope for mapping the stained cells.

From the immunocytochemical staining for the catecholaminergic rate-limiting synthesizing enzyme TH, it was found that the HUMSCs were TH positive following incubation with NCM for 6 days and then SHH and FGF8 in 10% FBS DMEM for 3 days. The HUMSCs were also TH positive following NCM for 6 days, and then SHH and FGF8 in NCM for 3 days (data not shown). However, the HUMSCs were not TH positive after a 6-day NCM incubation only. The double staining of human-specific nuclear antigen and DBH or GAD indicated that the HUMSCs were also differentiated into norepinephrine and GABAergic neurons in addition to the dopaminergic neurons.

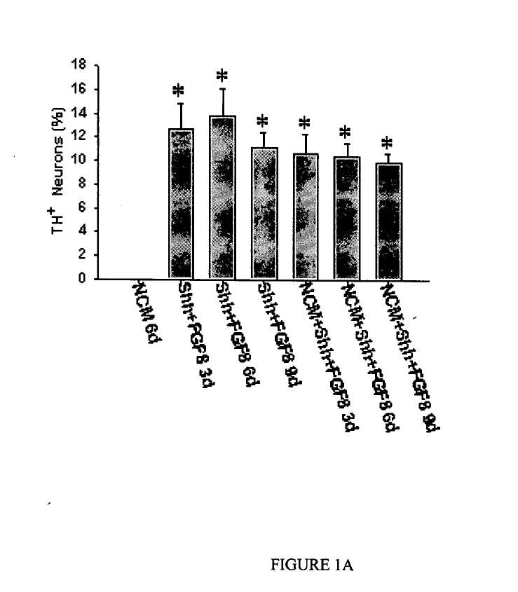

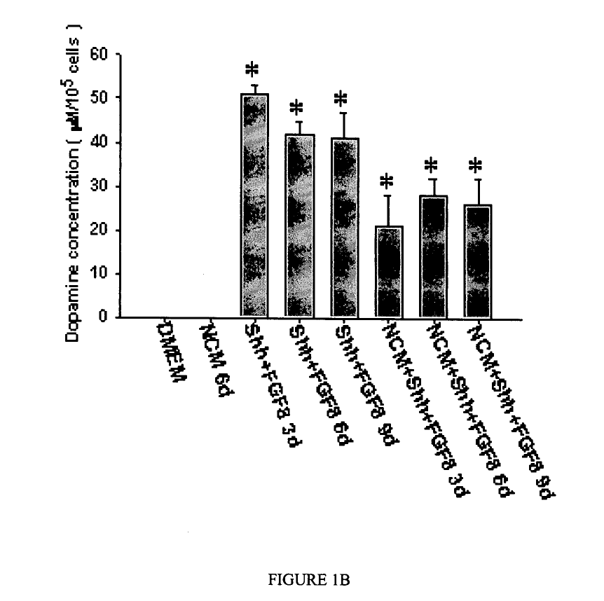

Referring to FIG. 1A, the proportion of TH-expressing neurons after treatment with NCM for 6 days followed by SHH and FGF8 for 3 days, was 12.7±2.1% (p<0.01) compared with the control group treated with only NCM for 6 days. No further increase in percentage of the TH-expressing neurons was observed in cells treated with SHH and FGF8 for 6 or 9 days. No significant difference in the percentages of the TH-expressing neurons was observed between cells incubated in SHH and FGF8 in DMEM or NCM (p>0.05). However, TH was not detected in HUMSCs treated with NCM only. The expression of TH protein (68 kD) was detected in the cells treated with NCM for 6 days followed by treatment with SHH and FGF8 for 3 days.

Referring to FIG. 1B, dopamine was not detected in the culture medium of HUMSCs treated with DMEM or NCM alone. However, dopamine concentration in the culture medium rose to a concentration of 51.0±2.0 μM as assayed by HPLC electrochemical detection after the culture of 10⁵ cells was treated with NCM for 6 days followed by treatment with SHH and FGF8 for 3 days (p<0.01). Also, the bis-benzimide-labeled cells were found in the striatum at twenty weeks after transplantation. A large number of cells staining positively for TH were clearly identified around the implantation site. The TH-positive cells stained with both human-specific nuclear antigen and TH were derived from HUMSCs. In contrast, no TH-positive cell was detected in the brains of rats that received grafted cells treated with NCM only (data not shown).

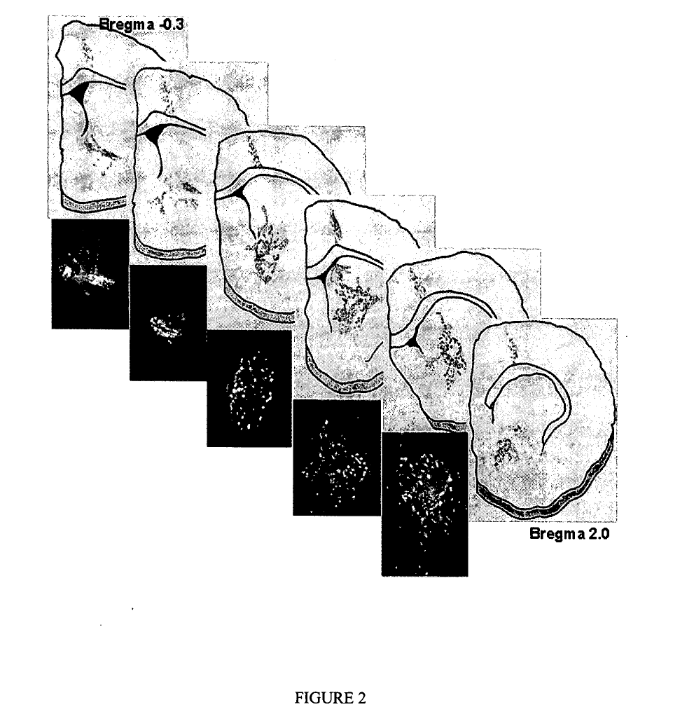

Cell migration patterns for the HUMSCs were observed with the aid of bis-benzimide labeling in 30 μm serial sections. Referring to FIG. 2, the labeled HUMSCs migrated for about 1.4 mm in both directions of the rostrocaudal axis from the implantation site (Bregma +1.0). Most of the labeled HUMSCs was localized in the region of Bregma +2.0 to the region of Bregma −0.3 (and even to −0.6 Bregma), almost throughout the entire striatum as shown in FIG. 2.

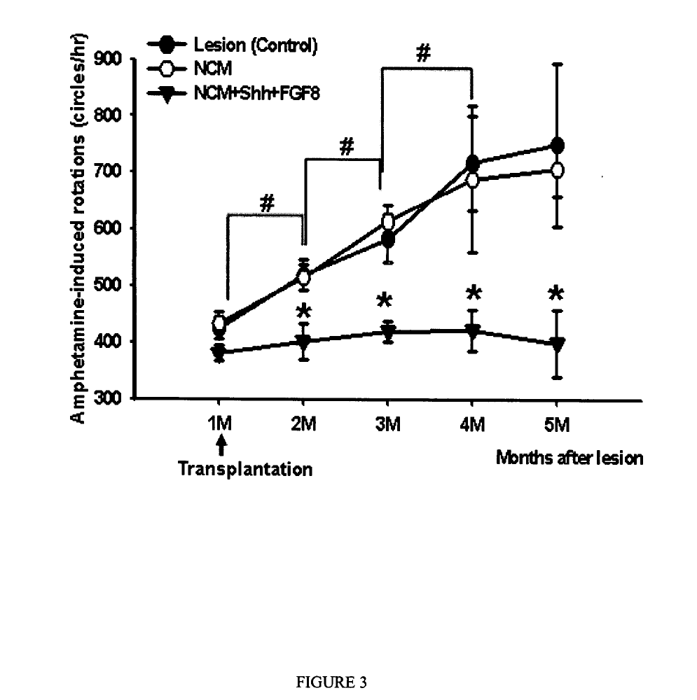

Referring to FIG. 3, the number of amphetamine-induced rotations in all groups reached 381.0±14.3 to 425.5±19.7 rotations per hour one month after 6-OHDA lesioning. The control group (n=12) which received injections of PBS in the dopamine-denervated striatum, showed a significant increase in number of the rotations. The increase in rotational scores was gradual over the months. The rats receiving graft cells treated with NCM only (NCM group) (n=12) showed no improvement (p>0.05). The rats receiving graft cells treated with NCM, SHH and FGF8 (NCM+SHH+FGF8 group) showed significantly less rotations than those in the control and NCM groups at the first observation period (1 month after lesion). Furthermore, while the rats of the control group and NCM group progressively exhibited more amphetamine-induced rotations over the next 4 months, the rats of the NCM+SHH+FGF8 group did not exhibit a significant increase in the rotations for the next 4 months.

### EXAMPLE 2

### Transplanting HUMSCs Transfected with Human Nurr1 Gene and Treated with SHH, FGF8 and NCM into the Brain of Parkinsonian Rat

### Isolation of Human Nurr1 RNA In Vivo

The differentiated human dopaminergic neurons were dissolved using 400 μl buffer RA1/4 μl β-ME (Sigma, St. Louis, Mo.) included in a NucleoSpin RNA II kit (BD Biosciences Clontech, Palo Alto, Calif.). The sample was triturated around 10 times using a size 21 needle and mixed with 250 μl 95% ethanol. Seven hundred μl of the sample mixture were transferred to the NucleoSpin column and centrifuged to remove waste at the bottom of the column. To each NucleoSpin column was then added 95 μl DNase I reaction mixture (90 μl DNase I reaction buffer and 10 μl reconstituted DNase) and the column was allowed to stand for 15 minutes. Five hundred μl of buffer RA2 were added to the NucleoSpin column and centrifuged to remove filtrate. The NucleoSpin column was placed in a 2 ml collection tube. Next, 600 μl of buffer RA3 was added to the NucleoSpin column and centrifuged to remove filtrate and the remaining solution. RNase free water was added to the NucleoSpin column to dissolve and elute human Nurr1 RNA.

### Cloning of Human Nurr1 Gene

A pCMV-HA vector (BD Biosciences Clontech, Palo Alto, Calif.) having a transgenic site modified to contain restriction sites Bgl II/Xho I was used for cloning human Nurr1 gene. The primers used in the reverse-transcription PCR were:

| | |
|---|---|
| Bgl II-Nurr1 | |
| 5′-GAAGATCTAGAGAGACGCGGAGAACTC-3′ of | SEQ ID NO: 2 |
| and | |
| Nurr1-Xho I | |
| 5′-CCGCTCGATTAGAAAGGTAAAGTGTC-3′ of. | SEQ ID NO: 3 |

The restriction enzymes Bgl II (New England Biolabs, Beverly, Mass.) and Xho I (New England Biolabs, Beverly, Mass.) recognized the DNA sequences of AGATCT and CTCGAG, respectively.

The Nurr1 cDNA fragment was amplified by reverse transcriptase polymerase chain reaction (RT-PCR) using M-MLV reverse transcriptase, RNase H minus, point mutant (Promega, Madison, Wis.) and Pfu DNA polymerase (Promega, Madison, Wis.) in a two-step procedure. In the reverse transcription step, 4 μl Nurr1 RNA and 1 μl Oligo (dT) 15 primer were transferred to an Eppendorf tube and heated to 70° C. in a PCR machine (Eppendorf® Mastercycler® personal) (Brinkmann Instruments, Westbury, N.Y.) for degrading the secondary structure of Nurr1 RNA. To the mixture were added 5 μM-MLV RT 5× reaction buffer, 1.25 μl PCR Nucleotide mix (Promega, Madison, Wis.), 0.75 μl RNase inhibitor (Invitrogen, Carlsbad, Calif.), 1 μl M-MLV RTase (Promega, Madison, Wis.) and topped up with RNase-free ddH₂O to 20 μl. The mixture was then subjected to thermal cycle reaction with the following conditions in the PCR machine (Eppendorf® Mastercycler® personal) (Brinkmann Instruments, Westbury, N.Y.).

| | |
|---|---|
| 40° C. | 10 min. |
| 50° C. | 50 min. |
| 70° C. | 15 min. |

In the PCR step, five μl of cDNA template were mixed with 5 μl Pfu DNA polymerase 10× buffer with MgSO₄, 1.25 μl PCR Nucleotide mix (Promega, Madison, Wis.), 3 μl upstream primer, 3 μl downstream primer, 1 μl Pfu DNA polymerase (Promega, Madison, Wis.) and topped up with ddH₂O to 50 μl. The mixture was subjected to 30 cycles of thermal cycle reaction with the following conditions in the PCR machine (Eppendorf® Mastercycler® personal) (Brinkmann Instruments, Westbury, N.Y.).

| | |
|---|---|
| 94° C. | 6 min. |
| 94° C. | 1 min. |
| 60° C. | 1 min. |
| 72° C. | 4 min. |
| 72° C. | 8 min. |
| 4° C. | overnight |

The PCR product was purified using QIAquick PCR purification kit (QIAGEN, Valencia, Calif.). The PCR product was mixed with buffer PB and transferred to a QIAquick spin column. The QIAquick spin column was centrifuged at the room temperature to remove filtrate at the bottom of the column. Seven hundred fifty μl of buffer PE were added to the column for washing DNA, and the column was centrifuged to remove filtrate and remaining ethanol. Next, the QIAquick spin column was placed in the Eppendorf tube, 50 μl elution buffer (10 mM Tris-HCl, pH 8.5) were added, the column stood for 2 minutes, and was centrifuged to collect filtrate containing DNA.

The Nurr1 cDNA was ligated to pCR-Blunt II-TOPO vector using topoisomerase, followed by digestion with the restriction enzyme to ensure Nurr1 cDNA having sticky ends at both ends for subsequent ligation. One μl of PCR products was added with 1 μl salt solution and 1 μl PCR II-Blunt-TOPO, topped up with ddH₂O to 50 μl, and mixed well using Zero Blunt® TOPO® PCR Cloning kit (Invitrogen, Carlsbad, Calif.). The mixture was allowed to stand in room temperature for 5 minutes for TOPO PCR cloning to take place.

### Transformation

A single colony of DH5a competent strain of E. coli was cultured in 5 ml LB broth and incubated in a 37° C. incubator for twelve hours. Two hundred μl of the bacterial solution was then subcultured in 5 ml LB broth and incubated in a 37° C. incubator for three hours until the OD₆₀₀ value reached between 0.2 and 0.4. The bacterial solution was then transferred to 50 ml centrifuge tube and placed on ice for 10 minutes, followed by centrifuging at 3,000 rpm for 15 minutes at 4° C. Next, the supernatant was removed, followed by adding ½ volume of TSS (1% NaCl, 1M MgSO₄, 10% PEG 4000, 5% DMSO, 5% yeast extract, and 1% tryptone) to 1 volume of the bacterial solution. The mixture was then vortexed and centrifuged at 300 rpm for 10 minutes at 4° C. The supernatant was removed and then 3.5 ml TSS-glycerol (50% TSS and 30% glycerol) were added. One hundred μl of the mixture was then transferred into a 1.5 ml centrifuge tube after mixing well and was stored at −80° C. for further use.

Five μl of vector or ligation products were mixed with DH5a competent strain of E. coli and chilled on ice for about 30 minutes. The mixture was then heat shocked in a 42° C. water bath and chilled on ice again. In a germ-free laminar flow, to the mixture was added 250 μl S.O.C. medium (Invitrogen, Carlsbad, Calif.) and incubated in a 37° C. incubator for one hour. One hundred μl of the bacterial solution was evenly spread on the LB agar plate (Pronadisa, Madrid, Spain) (10 g/L NaCl, 10 g/L Bacto-tryptone, 5 g/L yeast extract, 15 g/L agar, pH 7, and 100 μg/ml ampicillin or 50 μg/ml kanamycin (Sigma, St. Louis, Mo.)). The plate was placed in the incubator at 37° C. for culturing. Observations were made with respect to the growth of a semi-transparent colony. The plasmid of the differentiated colony was determined by PCR. The DNA sequence was then sent for sequencing (Genomics BioSci & Tech Co).

### Mini Plasmid Purification

A single colony was selected and cultured into 5 ml LB medium containing 100 μg/ml ampicillin or 50 μg/ml kanamycin (Sigma, St. Louis, Mo.) followed by culturing in an incubator at 37° C. for fourteen to sixteen hours. Five hundred μl of bacterial solution were transferred to a tube, along with 500 μl 60% glycerol and then stored at −80° C.

The amplified plasmid was isolated using QIAprep Spin Miniprep Kit (QIAGEN, Valencia, Calif.). The bacterial solution was transferred into a 1.5 ml microcentrifuge tube, followed by centrifuging at 6000 rpm for 1 minute to remove the supernatant. Two hundred fifty ml Buffer P1 were added to the pelleted bacterial cell and mixed well. Next, 250 μl Buffer P2 were added to the mixture and mixed thoroughly by inverting until the solution became viscous and slightly clear. Then, 350 μl Buffer P3 were added to the mixture, followed by immediately inverting the tube 4-6 times and then placing on ice for 10 minutes. The mixture was centrifuged at 12,500 rpm for 10 minutes. The supernatant was applied to a QIAprep spin column, followed by centrifuging at 12,500 rpm for 1 minute and discarding the flow-through. 0.5 ml Buffer PB was added to wash the QIAprep spin column, followed by centrifuging at 12,500 rpm to remove the flow-through, and finally 0.75 ml Buffer PB was added, followed by centrifuging at 12,500 rpm to further remove the flow-through. Then, the QIAprep spin column was placed in a clean microcentrifuge tube, followed by adding 50 μl Buffer EB to elute the DNA. After standing for 20 minutes and centrifuging at 12,500 for 1 minute, the plasmid DNA was obtained.

Digestion with Restrictive Enzyme

Ten μl of the vector DNA (pCMV-HA) (BD Biosciences Clontech, Palo Alto, Calif.) or PCR product (purified Nurr1 PCR product) were added with 2 μl 10×NEB buffer 2 (0.5 mM NaCl, 0.1 M Tris-HCl, 0.1M MgCl₂, 10 mM DTT, pH 7.9), 1 μg Bgl II/Xho I (New England Biolabs, Beverly, Mass.), topped up with ddH₂O to 20 μl, and stood in a 37° C. water bath for overnight reaction. The mixture was then stood in a 65° C. water bath for 20 minutes to deactivate the restrictive enzymes, and separated by gel electrophoresis. The gel electrophoresis was conducted using 1% LE agarose (CAMBREX, Rockland, Me.) to separate the ligation products only and the ligation products digested by the restrictive enzyme.

### Gel Extraction

The QIAquick Gel Extraction Kit (QIAGEN, Valencia, Calif.) was used here in gel extraction and purification. The target DNA fragments were excised from the agarose gel with a clean, sharp scalpel and then placed into a 1.5 ml centrifuge tube. The gel slice was weighted in a colorless tube, followed by adding 3 volumes of Buffer QG to 1 volume of gel. The tube was incubated at 50° C. in a water bath for 10 minutes and mixed by vortexing the tube every 2-3 minutes during the incubation until the gel has completely dissolved. One gel volume of isopropanol was added into the sample and mixed well. The sample was placed onto the QIAquick spin column, centrifuged at 13,000 rpm for 1 minute and the liquid which passed though the column was discarded. Then 0.5 ml Buffer QG was added to the sample and centrifuged at 13,000 rpm for 1 minute. Further, 0.75 ml Buffer PE was added to the sample and centrifuged at 13,000 rpm for 1 minute. The resting Buffer PE was finally removed by centrifugation at 13,000 rpm for 1 minute. The QIAquick spin column was transferred to a clean 1.5 ml microcentrifuge tube, followed by adding 50 μl Buffer EB onto the QIAquick membrane, standing for 3 minutes and centrifuging at the column at 13,000 rpm for 1 minute. The purified DNA was collected and stored at −20° C.

### Ligation

The Nurr1 was ligated to pCMV-HA by mixing pCMV-HA with Nurr1 in a concentration ratio of 1:3. To this mixture were added 2 μl 10× T4 DNA ligase buffer (0.5M Tris-HCl, 0.1 M MgCl₂, 0.1 M DTT, 10 mM ATP, 0.25 mg/ml BSA, pH 7.5), 1 μl T4 DNA ligase (New England Biolabs, Beverly, Mass.), topped up with ddH₂O to 20 μl, and mixed well. The mixture was then allowed to stand in a 16° C. water bath for overnight reaction.

### Electrophoresis and Sequencing

The ligation products only and the ligation products digested by the restrictive enzymes were identified by gel electrophoresis using 1% LE agarose (CAMBREX, Rockland, Me.). The ligation product with the correct size was submitted for sequencing (Genomics BioSci & Tech Co.), and the resulting nucleotide sequences were analyzed by BLAST provided by NCBI.

### Maxi Plasmid Purification

The amplified plasmid was isolated using EndoFree Plasmid Maxi Kits (QIAGEN, Valencia, Calif.) after transformation. Five ml of the overnight-cultured bacterial solution was transferred to 250 ml LB broth and cultured in the incubator at 37° C. for 12 hours. Next, the bacterial solution was centrifuged to remove supernatant. The cell pellet was re-suspended with 10 ml buffer P1. The cell suspension was mixed with 10 ml buffer P2, followed by mixing with 10 ml buffer P3 until a white precipitate was generated. The cell lysate was poured into a barrel of a QIA filter cartridge and allowed to stand at room temperature for 10 minutes. Next, the QIA filter cartridge outlet nozzle was removed to insert the push bar and collect the filtrate in a 50 ml conical tube. The filtrate was mixed with 2.5 ml buffer ER and chilled on ice. The QIAGEN-tip 500 was balanced with 10 ml buffer QBT, followed by pouring the sample from the conical tube to the QIAGEN-tip 500. Once the supernatant was filtered off, the QIAGEN-tip 500 was washed twice with 30 ml buffer QC. The DNA was then eluted with 15 ml buffer QN and isopropanol was added at room temperature to precipitate DNA. The DNA precipitate was centrifuged. The DNA pellet was re-suspended in 5 ml endotoxin-free 70% ethanol at room temperature, centrifuged to remove ethanol supernatant, and left in the hood for evaporating off the remaining ethanol. DNA was dissolved using 500 μl endotoxoin-free buffer TE. The light absorbance of the plasmid solution was measured at OD₂₆₀ using an optical spectrum analyzer. The concentration of plasmid DNA was adjusted to 1 μg/μl.

### Transient Transfection by Electroporation

The human Nurr1 gene was transiently transfected using a Human MSC Nucleofector™ kit (Amaxa biosystems, Cologne, Germany) to increase the amount of Nurr1 expression. Each tube having 5×10⁵ HUMSCs in trypsin-EDTA was prepared and centrifuged to remove supernatant, and the cell pellet was re-suspended with 100 μl Human MSC Nucleofector solution. Next, each tube was mixed with 2 μg pCMV-HA-Nurr1 or pCMV-HA (vehicle). The mixture was transferred into an amaxa cuvette, which in turn was placed in an electroporation instrument. A high transfection efficiency program U-23 was selected to initiate electroporation. The cuvette was removed from the electroporation instrument immediately after electroporation. Five hundred μl RPMI medium (Gibco, BRL, USA) were added to the cuvette. Next, the cells were sucked out of the cuvette and re-seeded in a culture dish supplemented with culture medium.

The HUMSCs transfected with the pCMV-HA-Nurr1 or pCMV-HA were further treated with FGF8, SHH and NCM by culturing the HUMSCs with a composition comprising the murine N-terminal fragment of SHH, and the murine FGF8 isoform b in the NCM, so that the HUMSCs are differentiated into dopaminergic neurons. Specifically, the HUMSCs transfected with the pCMV-Ha vector were divided into two groups. The first group was treated with NCM for 6 days, followed by treating with FGF8 and SHH for 3 days. The second group was treated with FGF8 and SHH for 3 days, followed by treating with NCM for 6 days. The HUMSCs transfected with the pCMV-Ha-Nurr1 vector were divided into four groups. The first group was treated with DMEM for 9 days. The second group was treated with NCM for 9 days. The third group was treated with NCM for 6 days, followed by treatment with FGF8 and SHH for 3 days. The fourth group was treated with FGF8 and SHH for 3 days, followed by treatment with NCM for 6 days.

### Quantification of the Human Nurr1 Gene by Real-Time PCR

The primers for real-time PCR were designed according to PRIMER EXPRESS software, version 1.5 (Applied Biosystem, Foster City, Calif.). The primers include:

| | |
|---|---|
| Nurr1-F | |
| 5′-AGTAGGGTCCTCGCCTCAA-3′ of | SEQ ID NO: 4 |
| Nurr1-R | |
| 5′-ATTCTCCCGAAGAGTGGTAACTGT-3′ of | SEQ ID NO: 5 |
| TH-F | |
| 5′-TGCCACGCTGTACTGGTTCAC-3′ of | SEQ ID NO: 6 |
| TH-R | |
| 5′-GTCGAAGGCCCGAATCTCA-3′ of | SEQ ID NO: 7 |
| AADC-F | |
| 5′-ACAGACTTAACGGGAGCCTTTAGA-3′ of | SEQ ID NO: 8 |
| AADC-R | |
| 5′-GTGATAAGCCCTGAATCCTGATG-3′ of | SEQ ID NO: 9 |
| DAT-F | |
| 5′-CATAGACGGCATCAGAGCATACC-3′ of | SEQ ID NO: 10 |
| DAT-R | |
| 5′-CCGCGTCAATCCAAACAGA-3′ of | SEQ ID NO: 11 |
| GAPDH-F | |
| 5′-TGGTATCGTGGAAGGACTCA-3′ of | SEQ ID NO: 12 |
| GADPH-R | |
| 5′-AGTGGGTGTCGCTGTTGAAG-3′ of | SEQ ID NO: 13 |

In the reverse transcription step, 4 μl Nurr1 RNA (Promega, Madison, Wis.) and 1 μl Oligo (dT) 15 primer (Promega, Madison, Wis.) were transferred to an Eppendorf tube and heated up to 70° C. in a PCR machine (Eppendorf® Mastercycler® personal) (Brinkmann Instruments, Westbury, N.Y.) for degrading the secondary structure of Nurr1 RNA. To the mixture were added 5 μl M-MLV RT 5× reaction buffer, 1.25 μl PCR Nucleotide mix (Promega, Madison, Wis.), 0.75 μl RNase inhibitor (Invitrogen, Carlsbad, Calif.), 1 μl M-MLV RTase (Promega, Madison, Wis.) and topped up with RNase-free ddH₂O to 20 μl. The mixture was then subjected to thermal cycle reaction in the PCR machine (Eppendorf® Mastercycler® personal) (Brinkmann Instruments, Westbury, N.Y.).

In the PCR step, diluted cDNA template was mixed with primer (0.6 mM), AmpliTaq Gold DNA polymerase, optimized buffer system and SYBR® green PCR master mix containing nucleotides (Applied Biosystem, Foster City, Calif.). Ten μl of the mixture were subjected to real-time PCR using an ABI Prism 7700 sequence detection system (Applied Biosystem, Foster City, Calif.).

### Preparation of Neuronal Conditional Medium

The Sprague-Dawley rats used in this experiment were anesthetized by intraperitoneal injection of 0.3 ml 10% chloride hydrate (Kanto Chemical, Kanto, Japan). The brain was removed, placed in a 15 ml centrifuge tube, and centrifuged at 90 g for 3 minutes. The resulting supernatant was removed, and then 1 ml 10% FBS-DMEM was added to break down the brain tissue. After mixing during and after adding 23 ml 10% FBS-DMEM, the mixture was randomly placed into coated 24-well culture plates and then incubated in a cell incubator. Then, 2 μl of 1 mM cytosine β-D-arabino furanoside (Sigma-Aldrich, St. Louis) was added into each well of the plates 24 hours later to inhibit the growth of glial cells. On the fifth day of culture, the culture medium was collected as neuronal conditioned medium (NCM) for use to culture umbilical mesenchymal stem cells. Five hundred ng/ml SHH, R&D Systems, Minneapolis, Minn.) and 100 ng/ml FGF8 (R&D Systems, Minneapolis, Minn.) were applied to treat these umbilical mesenchymal stem cells.

The Differentiation of the HUMSCs into Dopaminergic Neurons

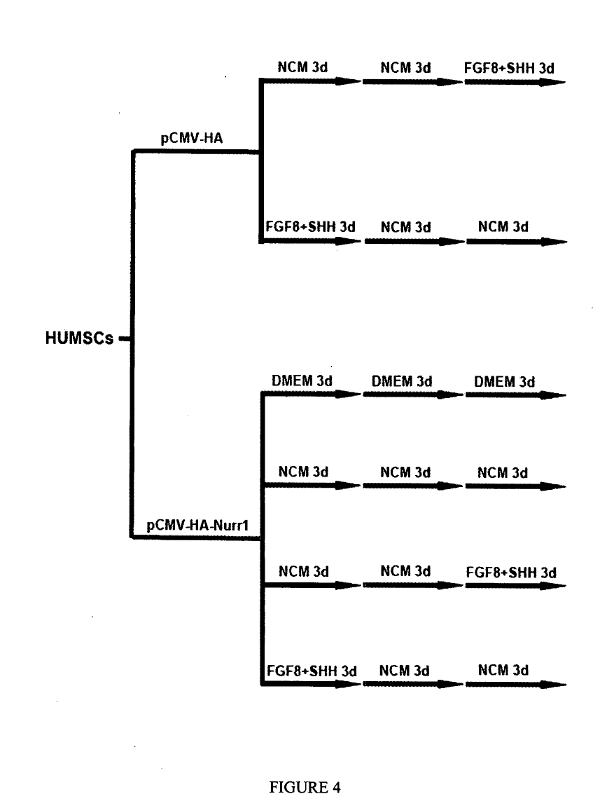

The HUMSCs were transfected with pCMV-HA or pCMV-HA-Nurr1 vector followed by treating with SHH and FGF8 and NCM for 9 days to allow differentiation of the HUMSCs into dopaminergic neurons expressing TH as shown in FIG. 4. The HUMSCs that were transfected with pCMV-HA were divided to two groups. The first group was treated with NCM for 6 days, followed by treating with FGF8+SHH for 3 days. The second group was treated with FGF8+SHH for 3 days, followed by treating with NCM for 6 days. Meanwhile, the HUMSCs that were transfected with pCMV-HA-Nurr1 were divided into four groups. The first group was treated with DMEM for 9 days, the second group was treated with NCM for 9 days, the third group was treated with NCM for 6 days, followed by treating with FGF8+SHH for 3 days, and the fourth group was treated with FGF8+SHH for 3 days, followed by treating with NCM for 6 days.

### HA-Taq Immunostaining

HUMSCs and brain sections of the rats were fixed with 4% paraformaldehyde in 0.1 M phosphate buffer for 20 min., and washed with 0.1 M phosphate buffer. The cells and brain sections were treated with a blocking solution (0.05% Triton X-100, 5% normal goat serum and 3% bovine serum albumin) for 30 min. in order to prevent nonspecific antibody-antigen binding. The cells and 10 brain sections were then reacted with rabbit anti-TA-Tag antibodies (BD Biosciences Clotech) at 4° C. for 18 h., and washed with 0.1 M PBS to remove any non-specific bindings. Next, cells and brain sections binding to the primary antibodies were reacted with secondary antibodies at room temperature for 1 h., and washed again with 0.1 M PBS to remove any non-specific bindings. The cell and brain sections binding to both primary and secondary antibodies were further reacted with ABC complex (ABC KIT, Vectorlabs PK-4000) at room temperature for 1 h., and washed with 0.1 M PBS to remove any non-specific bindings. The cells and brain sections binding to ABC complex were developed with 3,3′-diaminobenzidine (DAB) (5 mg DAB, 3.5 μl of 30% H₂O₂ in 10 ml of 50 mM Tris Buffer). Ethanol with increasing concentration, such as 50%, 70%, 80%, 90%, 95% and 100%, and xylene were applied in dehydration for 5 minutes. The cells and brain section were then observed under a microscope (Olympus BX-50).

### Immunocytochemistry for TH

HUMSCs and brain sections of the rats were fixed with 4% paraformaldehyde in 0.1 M phosphate buffer for 20 min., and washed with 0.1 M phosphate buffer. The cells and brain sections were treated with a blocking solution (0.05% Triton X-100, 5% normal goat serum and 3% bovine serum albumin) for 30 min. in order to prevent nonspecific antibody-antigen binding. The cells and brain sections were then reacted with mouse anti-TH antibodies (Chemicon, Temecula, Calif.) at 4° C. for 18 h, and washed with 0.1 M PBS to remove any non-specific bindings. Next, cells and brain sections binding to the primary antibodies were reacted with secondary antibodies at room temperature for 1 h., and washed again with 0.1 M PBS to remove any non-specific bindings. The cell and brain sections binding to both primary and secondary antibodies were further reacted with ABC complex (ABC KIT, Vectorlabs PK-4000) at room temperature for 1 h., and washed with 0.1 M PBS to remove any non-specific bindings. The cells and brain sections binding to ABC complex were developed with 3,3′-diaminobenzidine (DAB) (5 mg DAB, 3.5 μl of 30% H₂O₂ in 10 ml of 50 mM Tris Buffer).

### Transplantation of Dopaminergic Neurons

The HUMSCs were transfected with pCMV-HA or pCMV-HA-Nurr1 vector and treated with SHH, FGF8 and NCM to allow differentiation of the HUMSCs into dopaminergic neurons expressing TH. Next, the neurons were labeled with 1 μg/ml bis-benzimide (Sigma, St. Louis, Mo.) for 48 hours. The neurons were treated with trypsin for 3 minutes. The cell solution was collected and centrifuged. The cell pellet was re-suspended with DMEM added into the cell solution. The cell solution containing 1×10⁵ to 4×10⁵ neurons was microinjected into the left striatum (AP: 0.0 mm, R/L: +3.0 mm, H: −6.0 mm) of the Parkinsonian rats prepared according to Example 1.

Behavior Test for Parkinsonian Rats Grafted with Dopaminergic Neurons

The rats were divided into four groups, with each group containing 6 rats. One month after 6-OHDA injection, each of the rats in group 1 (control) was injected with phosphate-buffered saline (PBS) into their dopamine-denervated striata. Each of the rats in group 2 (CMV-HA+FGF8+SHH+NCM (4×10⁵)) was grafted with a suspension of 4×10⁵ HUMSCs that had been transfected with pCMV-HA vector and cultured in the FGF8 and SHH for 3 days, followed by NCM for 6 days. Each of the rats in group 3 (CMV-HA-Nurr1+FGF8+SHH+NCM (1×10⁵)) was grafted with a suspension of 1×10⁵ HUMSCs that had been transfected with pCMV-HA-Nurr1 vector and cultured in FGF8 and SHH for 3 days, followed by NCM for 6 days. Each of the rats in group 4 (CMV-HA-Nurr1+FGF8+SHH+NCM (4×10⁵)) was grafted with a suspension of 4×10⁵ HUMSCs that had been transfected with pCMV-HA-Nurr1 vector and cultured in FGF8 and SHH for 3 days, followed by NCM for 6 days. The effects of stem cell transplantation were examined in 6-OHDA-lesioned animals by quantification of rotations in response to amphetamine. Rotational scores were examined at 1, 2, 3 and 4 months post-transplantation. All behavioral tests were performed in a closed room to avoid any environmental disturbance and assessed by an independent observer blinded to the treatments.

### Positron Emission Tomography (PET)

The radioisotope used in PET study or diagnosis of Parkinson's disease mainly involves ¹⁸F which is positron emitting and has a half-life of 109.7 minutes. The radioisotope ¹⁸F may be linked to a benzyl ring of L-DOPA by chemical synthesis to form 6-[¹⁸F] fluoro-L-DOPA which shares the same biochemical metabolic pathway with L-DOPA and competes with L-DOPA for aromatic L-amino acid decarboxylase (AADC). Besides, 6-[¹⁸F] fluoro-L-DOPA can penetrate the blood-brain barrier to enter the dopaminergic neurons of the striatum. 6-[¹⁸F] Fluoro-L-DOPA is decarboxylated with aromatic L-amino acid decarboxylase in the neurons to form 6-[¹⁸F] fluoro-L-dopamine.

An inhibitor, carbidopa (28 mg/kg in saline), was given by intraperitoneal injection to the Parkinsonian rat to suppress AADC from the peripheral tissue and reduce the metabolism of 6-[¹⁸F] fluoro-L-DOPA in the peripheral tissue, so as to improve bioavailability of 6-[¹⁸F] fluoro-L-DOPA. After the rat was treated with the inhibitor for 30 minutes, the rat was given 6-[¹⁸F] fluoro-L-DOPA (mCi/kg) by intravenous injection. PET was conducted to monitor the absorption of ¹⁸F-DOPA in the striatum.

### Statistical Analyses

All data were presented as mean±standard error. One-way or two-way analysis of variance was used to compare all means, and least-significant difference was used for the posteriori test. In all statistical analyses, p<0.05 was considered significant.

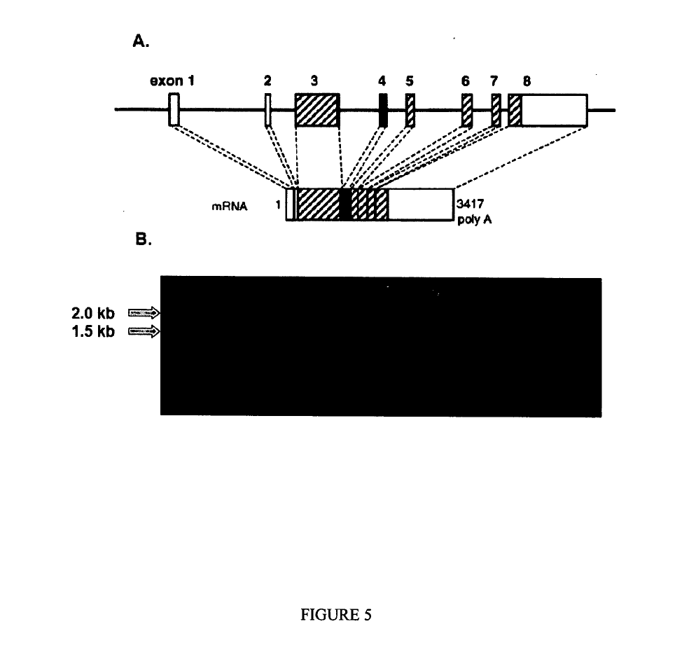

Referring to FIG. 5A, the human Nurr1 gene consists of 8 exons. The diagonal parts represent the coding regions, while the resting parts represent the uncoding regions. The total length of e human Nurr1 mRNA is 1797 bp and the successfully cloned Nurr1 cDNA fragments are shown in FIG. 5B (1797 bp).

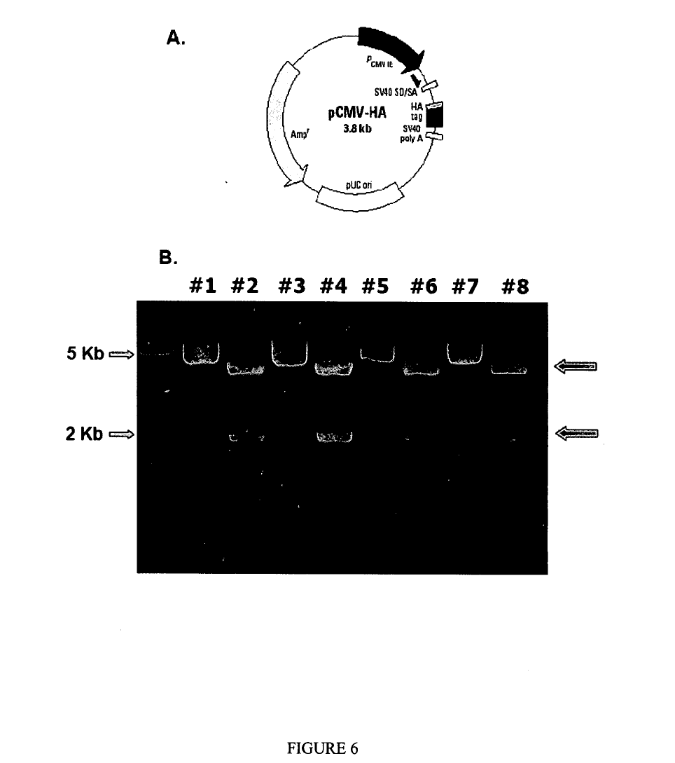

Referring to FIG. 6A, the Nurr1 cDNA was constructed into pCMV-HA vector. Referring to FIG. 6B, the pCMV-HA-Nurr1 vector with 5.6 kb is shown (#1, #3, #5 and #7); after restriction enzyme treatment, the empty pCMV-HA vector with 3.8 kb and Nurr1 cDNA only with 1.8 kb are shown (#2, #4, #6 and #8).

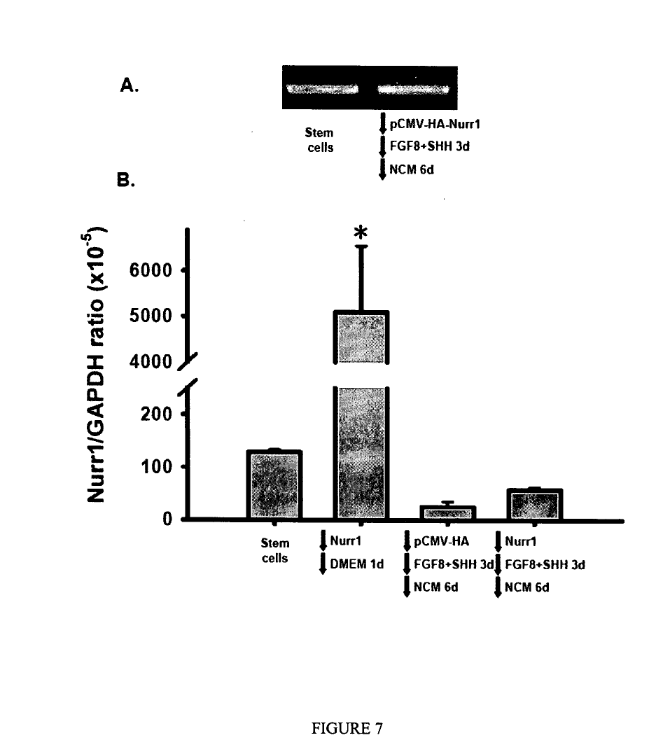

Referring to FIG. 7A, the Nurr1 gene expression of HUMSCs only and HUMSCs transfected with Nurr1 gene measured by real-time PCR are shown. Referring to FIG. 7B, the Nurr1 gene expression quantified by real-time PCR in the HUMSCs transfected with Nurr1 gene was at least 40 times higher than the HUMSCs or HUMSCs transfected with pCMV-HA.

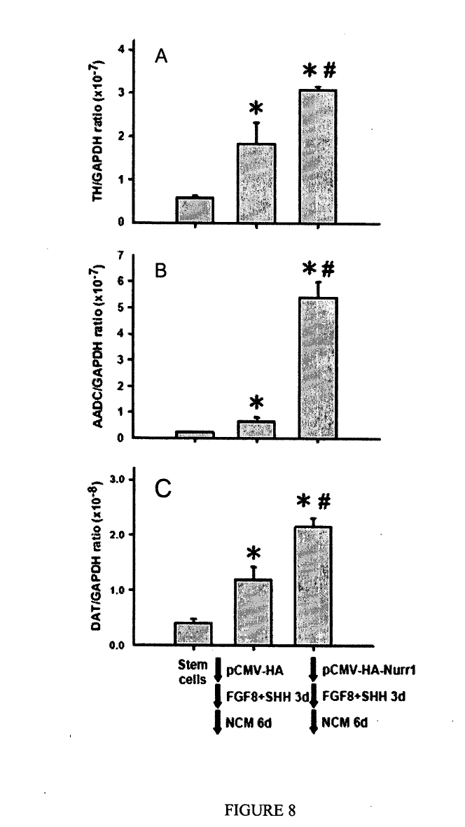

Referring to FIG. 8A, the expression of TH mRNA measured in terms of TH/GADPH ratio was significantly higher (p<0.05) in the HUMSCs transfected with pCMV-HA or pCMV-HA-Nurr1 vector and treated with FGF8 and SHH for 3 days, and NCM for 6 days, than the HUMSCs without any treatment. Referring to FIG. 8B, expression of AADC mRNA measured in terms of AADC/GADPH ratio was significantly higher (p<0.05) in the HUMSCs transfected with pCMV-HA-Nurr1 vector and treated with FGF8 and SHH for 3 days, and NCM for 6 days, than both the HUMSCs transfected with pCMV-HA vector and HUMSCs without any treatment. As shown in FIG. 8C, the expression of DAT mRNA measured in terms of DAT/GADPH ratio was significantly higher (p<0.05) in the HUMSCs transfected with pCMV-HA or pCMV-HA-Nurr1 vector and treated with FGF8 and SHH for 3 days, and NCM for 6 days, than the HUMSCs without any treatment. Also, from FIGS. 8A to 8C, it was found that the HUMSCs transfected with pCMV-HA-Nurr1 vector expressed higher TH, AADC and DAT mRNA than the HUMSCs transfected with pCMV-HA vector. Therefore, as the HUMSCs were induced to differentiate into the neurons by transfecting with the pCMV-HA-Nurr1 vector and treatment with FGF8, SHH and NCM, expression of the genes related to dopamine synthesis was up-regulated.

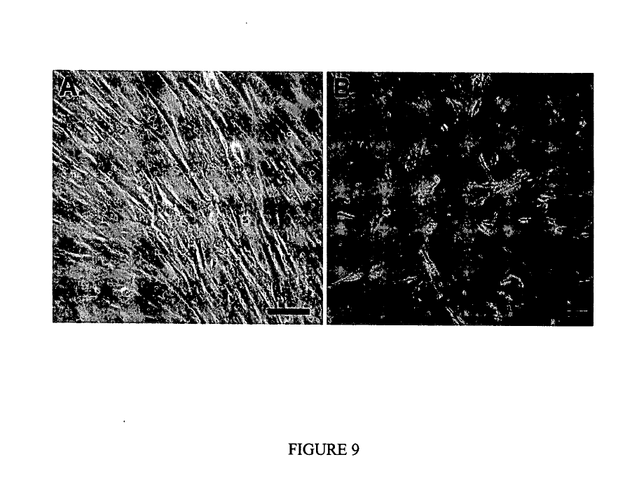

Referring to FIG. 9A, as the HUMSCs were cultured in 10% FBS-DMEM for 9 days, the cells were differentiated from mesenchymal-like shape into fibroblast-like morphology. In contrast, the HUMSCs were differentiated into cells that displayed neuronal-like morphology associated with shrinkage of cell bodies and fine cellular projections when the HUMSCs were transfected with pCMV-HA-Nurr1 vector and treated with NCM, FGF8 and SHH for a combined total of 9 days (FIG. 9B).

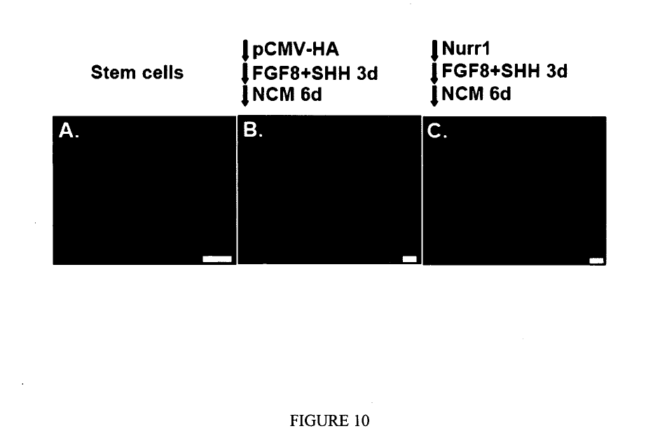

Referring to FIG. 10A, there were few TH positive neurons found from the HUMSCs without any treatment. Referring to FIG. 10B, there were about 25% TH positive neurons found from the HUMSCs, as the HUMSCs were transfected with pCMV-HA vector and treated with NCM for 6 days, followed by FGF8 and SHH for 3 days. Further, referring to FIG. 10C, the there were about 70% TH positive neurons found from the HUMSCs, as the HUMSCs were transfected with pCMV-HA-Nurr1 vector and treated with FGF8 and SHH for 3 days, followed by NCM for 6 days.

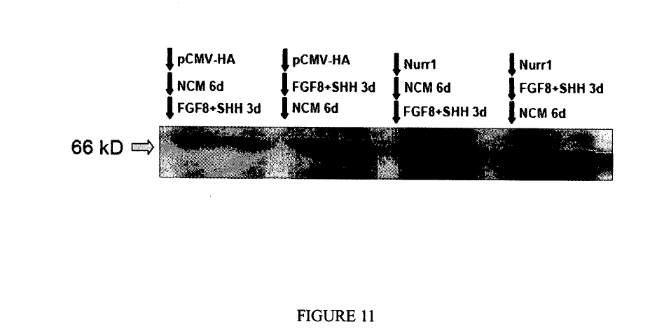

Referring to FIG. 11, cellular expression of TH was found from the HUMSCs, as the HUMSCs were transfected with pCMV-HA vector and treated with NCM for 6 days, followed by FGF8 and SHH for 3 days or treated with FGF8 and SHH for 3 days, followed by NCM for 6 days. Moreover, the cellular expression of TH in HUMSCs transfected with pCMV-HA-Nurr1 was significantly higher than that transfected with pCMV-HA vector, either treated with NCM for 6 days, followed by FGF8 and SHH for 3 days or treated with FGF8 and SHH for 3 days, followed by NCM for 6 days.

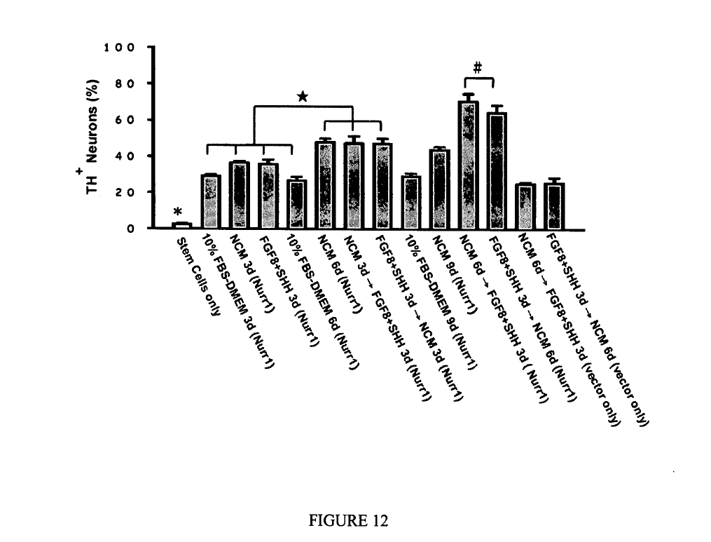

Referring to FIG. 12 and Table 1, the percentage of expression of TH positive neurons was measured. There were only 2.5% of TH positive neurons found from the HUMSCs without any treatment. As the HUMSCs were transfected with pCMV-HA vector and treated with NCM for 6 days, followed by FGF8 and SHH for 3 days, there were 25.05±0.74% TH positive neurons found from the HUMSCs. A similar result (25.46±2.96% TH positive neurons) was found when the HUMSCs were transfected with pCMV-HA vector and treated with FGF8 and SHH for 3 days, followed by NCM for 6 days. However, the percentage of TH positive neurons was much higher when the HUMSCs were transfected with pCMV-HA-Nurr1 vector and treated with NCM for 6 days, followed by FGF8 and SHH for 3 days (70.49±4.34% TH positive neurons) or when the HUMSCs were transfected with pCMV-HA-Nurr1 vector and treated with FGF and SHH for 3 days, followed by NCM for 6 days (64.29±4.12% TH positive neurons).

TABLE 1
The percentage of TH positive neuron differentiated from HUMSCs

| Groups | Mean ± S.E.M (%) |
|---|---|
| Stem cell only | 2.500 ± 0.306* |
| Nurr1 10% FBS-DMEM 3 d | 29.187 ± 0.592 |
| NCM 3 d (Nurr1) | 36.400 ± 0.941 |
| FGF8 + SHH 3 d (Nurr1) | 35.770 ± 2.438 |
| 10% FBS-DMEM 6 d | 26.720 ± 1.902 |
| NCM 6 d ((Nurr1) | 48.070 ± 1.847 ★ |
| NCM 3 d → FGF8 + SHH 3 d (Nurr1) | 47.373 ± 3.855 ★ |
| FGF8 + SHH 3 d →NCM 3 d (Nurr1) | 47.083 ± 2.923 ★ |
| 10% FBS-DMEM 9 d (Nurr1) | 29.245 ± 1.641 |
| NCM 9 d (Nurr1) | 44.027 ± 1.355 |
| NCM 6 d → FGF8 + SHH 3 d (Nurr1) | 70.493 ± 4.340 |
| FGF8 + SHH 3 d → NCM 6 d (Nurr1) | 64.290 ± 4.117 # |
| NCM 3 d → FGF8 + SHH 3 d (G418) | 72.903 ± 2.317 # |
| FGF8 + SHH 3 d → NCM 6 d | 70.628 ± 5.173 |
| NCM 6 d →FGF8 + SHH 3 d (vector only) | 25.047 ± 0.736 |
| FGF8 + SHH 3 d → NCM 6 d (vector only) | 25.463 ± 2.958 |

*P < 0.05, stem cell vs. the others;
★ p < 0.05 NCM 6 d, NCM + FGF8 + SHH 6 d and FGF8 + SHH + NCM 6 d vs. DMEM 3 d, NCM 3 d, FGF8 + SHH 3 d and DMEM 6 d;
# p < 0.05, NCM + FGF8 + SHH 9 d vs. the others, n = 4, one way ANOVA followed by LSD test; d = days.

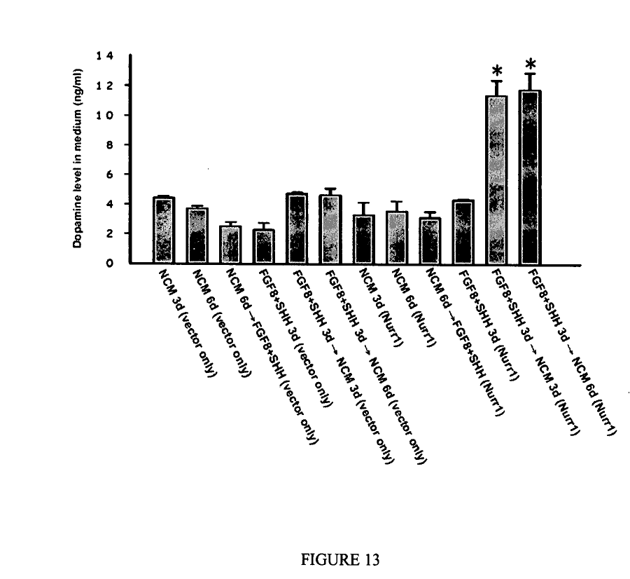

The dopamine concentration in the culture medium was assayed by HPLC analysis. There was almost no dopamine detected in the culture medium when the HUMSCs were not subjected to any treatment. However, the dopamine level in the medium was 2.28±0.46 to 4.74±0.08 ng/ml/10⁵ cells when the HUMSCs were transfected with pCMV-HA vector and treated with at least one of NCM, FGF8 and SHH as shown in FIG. 13 and Table 2. The dopamine level in the medium increased to about 11.39±1.02 and 11.78±1.12 ng/ml/10⁵ cells when the HUMSCs were transfected with pCMV-HA-Nurr1 vector and treated with FGF8 and SHH for 3 days, followed by NCM for 3 and 6 days. The serotonin level in the medium was also measured as shown in Table 3.

TABLE 2
The dopamine level secreted by the HUMSCs transfected with vector or
Nurr1 as the HUMSCs were subject to different treatments.

| Groups | Mean ± S.E.M (%) |
|---|---|
| Stem cell only | 0.013 ± 0.003 |
| NCM 3 d (vector only) | 4.423 ± 0.129 |
| NCM 6 d (vector only) | 3.713 ± 0.167 |
| NCM 6 d → FGF8 + SHH 3 d (vector only) | 2.513 ± 0.298 |
| NCM 6 d → FGF8 + SHH 3 d (vector only) in cells | 1.030 ± 0.271 |
| FGF8 + SHH 3 d (vector only) | 2.283 ± 0.464 |
| FGF8 + SHH 3 d → NCM 3 d (vector only) | 4.737 ± 0.081 |
| FGF8 + SHH 3 d →NCM 6 d (vector only) | 4.640 ± 0.447 |
| FGF8 + SHH 3 d →NCM 6 d (vector only) in cells | 1.143 ± 0.416 |
| NCM 3 d (Nurr1) | 3.310 ± 0.847 |
| NCM 6 d (Nurr1) | 3.560 ± 0.691 |
| NCM 6 d → FGF8 + SHH 3 d (Nurr1) | 3.123 ± 0.401 |
| NCM 6 d → FGF8 + SHH 3 d (Nurr1) in cells | 2.290 ± 0.082 |
| FGF8 + SHH 3 d (Nurr1) | 4.333 ± 0.073 |
| FGF8 + SHH 3 d → NCM 3 d (Nurr1) | 11.393 ± 1.020 |
| FGF8 + SHH 3 d → NCM 6 d (Nurr1) | 11.780 ± 1.123 |
| FGF8 + SHH 3 d → NCM 6 d (Nurr1) in cells | 2.583 ± 0.180 |
| NCM 3 d (G418) | 8.250 ± 3.017 |
| NCM 6 d (G418) | 12.270 ± 0.812 |
| NCM 6 d (G418) → FGF8 + SHH 3 d (G418) | 4.668 ± 0.591 |
| NCM 6 d (G418) → FGF8 + SHH 3 d (G418) in | 4.570 ± 1.484 |
| cells | |
| FGF8 + SHH 3 d (G418) | 6.393 ± 1.834 |
| FGF8 + SHH 3 d → NCM 3 d (G418) | 8.560 ± 1.142 |
| FGF8 + SHH 3 d → NCM 6 d (G418) | 8.507 ± 1.390 |
| FGF8 + SHH 3 d → NCM 6 d (G418) in cells | 4.293 ± 0.845 |

TABLE 3
The serotonin level secreted by the HUMSCs transfected with
vector or Nurr1 as the HUMSCs were subject to different treatments.

| Groups | Mean ± S.E.M (%) |
|---|---|
| Stem cell only | 0.523 ± 0.172 |
| NCM 3 d (vector only) | 0.918 ± 0.436 |
| NCM 6 d (vector only) | 4.943 ± 4.556 |
| NCM 6 d → FGF8 + SHH 3 d (vector only) | 10.757 ± 1.063 |
| NCM 6 d → FGF8 + SHH 3 d (vector only) in cells | 7.047 ± 2.6 |
| FGF8 + SHH 3 d (vector only) | 3.980 ± 1.457 |
| FGF8 + SHH 3 d → NCM 3 d (vector only) | 2.477 ± 1.494 |
| FGF8 + SHH 3 d →NCM 6 d (vector only) | 2.413 ± 1.661 |
| FGF8 + SHH 3 d →NCM 6 d (vector only) in cells | 9.363 ± 3.774 |
| NCM 3 d (Nurr1) | 1.137 ± 0.947 |
| NCM 6 d (Nurr1) | 2.310 ± 1.462 |
| NCM 6 d → FGF8 + SHH 3 d (Nurr1) | 6.357 ± 2.198 |
| NCM 6 d → FGF8 + SHH 3 d (Nurr1) in cells | 1.317 ± 0.676 |
| FGF8 + SHH 3 d (Nurr1) | 6.080 ± 5.415 |
| FGF8 + SHH 3 d → NCM 3 d (Nurr1) | 2.620 ± 1.746 |
| FGF8 + SHH 3 d → NCM 6 d (Nurr1) | 3.187 ± 2.663 |
| FGF8 + SHH 3 d → NCM 6 d (Nurr1) in cells | 22.003 ± 11.829 |
| NCM 3 d (G418) | 2.130 ± 0.318 |
| NCM 6 d (G418) | 1.860 ± 0.325 |
| NCM 6 d (G418) → FGF8 + SHH 3 d (G418) | 15.767 ± 3.616 |
| NCM 6 d (G418) → FGF8 + SHH 3 d (G418) in | 35.933 ± 11.202 |
| cells | |
| FGF8 + SHH 3 d (G418) | 0.943 ± 0.231 |
| FGF8 + SHH 3 d → NCM 3 d (G418) | 0.987 ± 0.242 |
| FGF8 + SHH 3 d → NCM 6 d (G418) | 0.857 ± 2.233 |
| FGF8 + SHH 3 d → NCM 6 d (G418) in cells | 20.810 ± 8.385 |

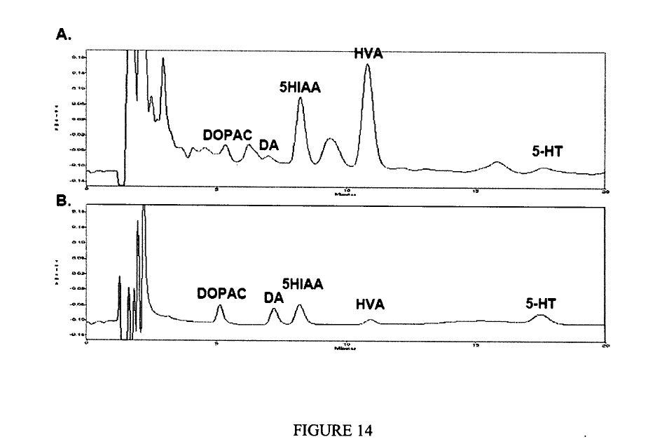

In addition, from the HPLC spectrum shown in FIG. 14A, it was found that dopamine (DA) and its metabolites such as 3,4-dihydroxyphenylacetic acid (DOPAC) and homovanillic acid (HVA) were found in the culture medium when the HUMSCs were transfected with the pCMV-HA-Nurr1 vector and treated with FGF8 and SHH for 3 days, and NCM for 6 days. Serotonin (5-HT) and its metabolites such as 5-hydroxyindoleacetic acid (5HIAA) were also found in the culture medium when the HUMSCs were transfected with the pCMV-HA-Nurr1 vector and treated with FGF8 and SHH for 3 days, followed by NCM for 6 days. From the HPLC spectrum shown in FIG. 14B, it was found that DA and its metabolites such as DOPAC and less HVA were found in the culture medium when the HUMSCs were transfected with the pCMV-HA vector and treated with FGF8 and SHH for 3 days, and NCM for 6 days. Less serotonin (5-HT) and its metabolites such as 5HIAA were also found in the culture medium when the HUMSCs were transfected with the pCMV-HA vector and treated with FGF8 and SHH for 3 days, followed by NCM for 6 days.

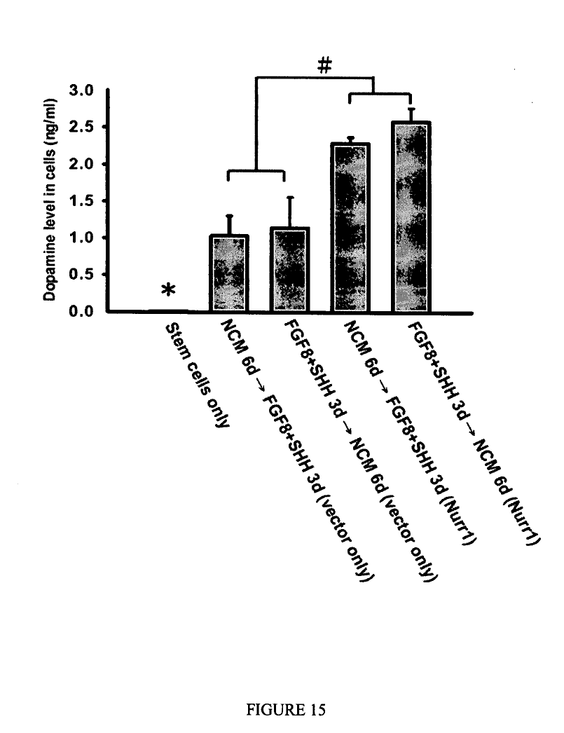

Referring to FIG. 15, the dopamine level detected in the HUMSCs without any treatment was 0.013±0.003 ng/ml/10⁵ cells. As the HUMSCs were transfected with pCMV-HA vector and treated with NCM, FGF8 and SHH, the dopamine level in the cells was 1.03±0.27 and 1.14±0.42 ng/ml/10⁵ cells as shown in FIG. 15. The dopamine level in the medium increased to about 2.29±0.08 and 2.58±0.18 ng/ml/10⁵ cells when the HUMSCs were transfected with pCMV-HA-Nurr1 vector and treated with FGF8 and SHH for 3 days, and NCM for 6 days or when the HUMSCs were transfected with pCMV-HA-Nurr1 vector and treated with NCM for 6 days, followed by FGF8 and SHH for 3 days.

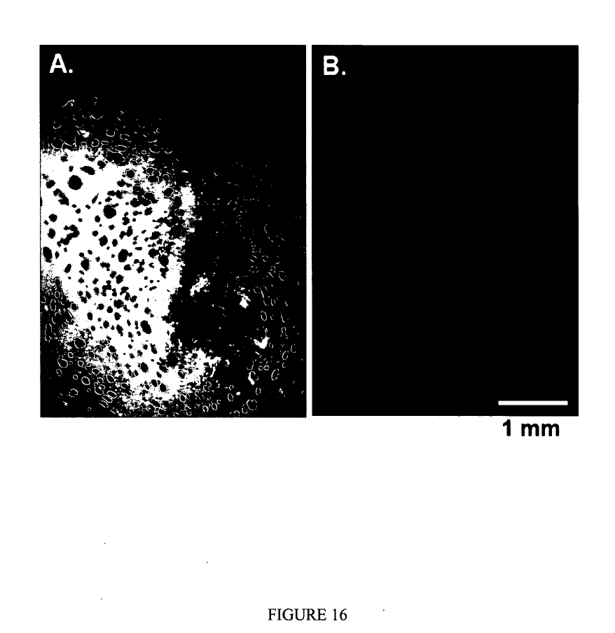

After the HUMSCs were transfected with pCMV-HA-Nurr1 vector and treated with FGF8 and SHH for 3 days, followed by NCM for 6 days, the nuclei of these HUMSCs were marked by bis-benzimide, and these HUMSCs were transplanted into the rats with Parkinson's disease. Four months later, a great amount of cells with bis-benamide was found at the implication site as shown in FIG. 16.

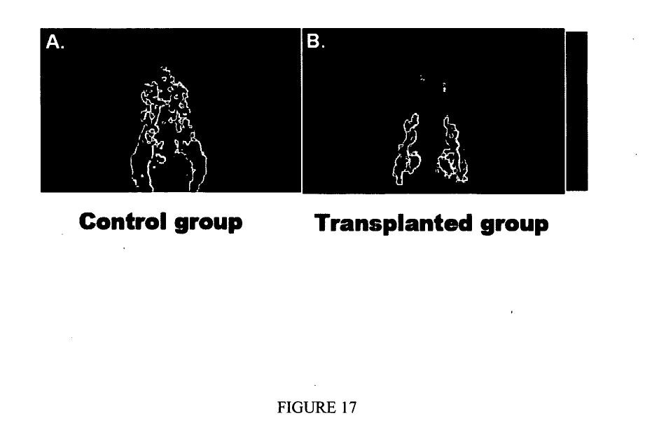

Referring to FIG. 17A, the micro-PET analysis has indicated no ¹⁸F-dopamine detected at the injured right striatum in the rat of the control group. In contrast, the rat of the CMV-HA-Nurr1+FGF8+SHH+NCM (4×10⁵) group has displayed the presence of ¹⁸F-dopamine at the injured right striatum (FIG. 17B). Accordingly, the cells grafted into the brain of the rat are dopaminergic neurons.

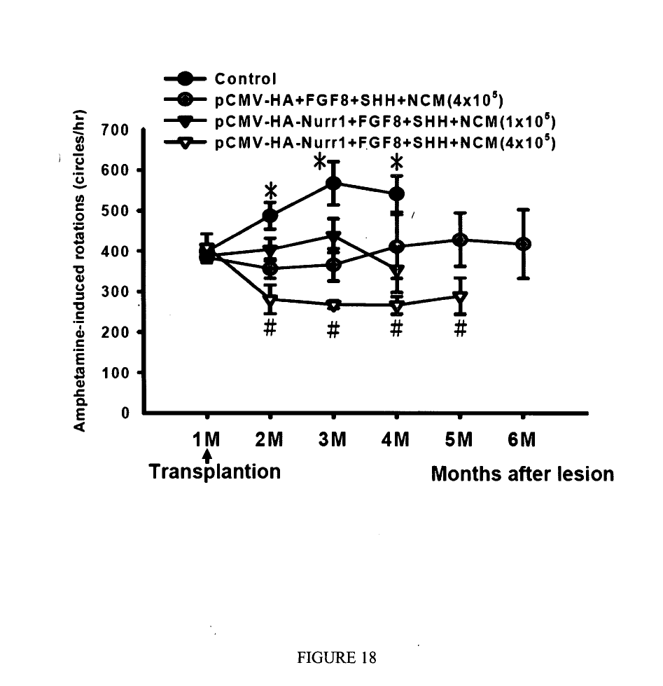

Referring to FIG. 18, the number of amphetamine-induced rotations in all groups reached 384.00±11.15 to 409.00±33.60 rotations per hour one month after 6-OHDA lesioning. The control group which received injections of PBS in the dopamine-denervated striatum, showed a significant increase in number of rotations over two to four months after 6-OHDA lesioning. No rats of CMV-HA+FGF8+SHH+NCM (4×10⁵) or CMV-HA-Nurr1+FGF8+SHH+NCM (1×10⁵) group showed any significant improvement (p>0.05) in the number of amphetamine-induced rotations. The number of amphetamine-induced rotations were 289.00±44.43 to 266.33±21.67 rotations per hour in the rats of CMV-HA-Nurr1+FGF8+SHH+NCM (4×10⁵) group one to four months after the cells were grafted. Therefore, the rats of CMV-HA-Nurr1+FGF8+SHH+NCM (4×10⁵) group showed significant reduction in number of amphetamine-induced rotations compared to the rats of the control group (Table 4).

TABLE 4
Rotation of individual rats in response to amphetamine tested at
1, 2, 3, 4, 5 and 6 months after lesioning

| | | Rotation No. | | | | | |
|---|---|---|---|---|---|---|---|
| | | | 2 M | 3 M | 4 M | 5 M | 6 M |
| | | 1 M after | after | after | after | after | after |
| Grouping | No. | lesion | lesion | lesion | lesion | lesion | lesion |
| Control | 44 | 397 | 400 | 480 | 521 | — | — |
| | 45 | 400 | 559 | 718 | 671 | — | — |
| | 48 | 390 | 486 | 504 | 508 | — | — |
| | 51 | 412 | 486 | 567 | 465 | — | — |
| pCMV + HA + | 18 | 380 | 401 | 296 | 273 | 296 | 254 |
| FGF8 + SHH + | 20 | 405 | 321 | 435 | 546 | 491 | 540 |
| NCM (4 × 10⁵) | 24 | 367 | 346 | 366 | 414 | 498 | 458 |
| pCMV + HA + | 34 | 421 | 403 | 406 | 357 | — | — |
| Nurr1 + FGF8 + | 35 | 365 | 453 | 387 | 255 | — | — |
| SHH + NCM (1 × | 59 | 378 | 355 | 521 | 450 | — | — |
| 10⁵) | | | | | | | |
| pCMV + HA + | 23 | 380 | 272 | 255 | 289 | 275 | — |
| Nurr1 + FGF8 + | 25 | 476 | 346 | 263 | 287 | 372 | — |
| SHH + NCM (4 × | 29 | 371 | 224 | 285 | 223 | 220 | — |
| 10⁵) | | | | | | | |

According to the present invention, HUMSCs are isolated and differentiated into dopaminergic, norepinephrine and GABAergic neurons in vitro. The neurons are transplanted into the brain of the mammal to treat the neurodegenerative diseases associated with loss of neurons synthesizing dopamine, norepinephrine and GABA. Specifically, when the dopaminergic neurons expressing TH are transplanted into the brain of the mammal, the syndrome of Parkinson's disease associated with loss of dopamine in the mammal is alleviated.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A cell system for treating a neurodegenerative disorder in a mammal, comprising a population of neurons differentiated from umbilical mesenchymal stem cells for expressing at least one of tyrosine hydroxylase (TH), dopamine-p-hydroxylase (DBH), glutamate decarboxylase (GAD), aromatic L-amino acid decarbxylase (AADC) and dopaminergic transporter (DAT) in a cell culture.

2. The cell system according to claim 1, wherein the umbilical mesenchymal stem cells are isolated from Wharton's jelly of a human umbilical cord.

3. The cell system according to claim 2, wherein the umbilical mesenchymal stem cells are differentiated by culturing with a composition comprising a mammalian hedgehog protein and mammalian fibroblast growth factor (FGF) in a culture medium.

4. The cell system according to claim 3, wherein the mammalian hedgehog protein includes a murine N-terminal fragment of sonic hedgehog protein.

5. The cell system according to claim 3, wherein the mammalian FGF includes a murine FGF8 isoform b.

6. The cell system according to claim 3, wherein the culture medium is neuronal conditioned medium (NCM).

7. The cell system according to claim 3, wherein the culture medium is Dulbecco's Modified Eagle's Medium (DMEM) supplemented with 10% fetal bovine serum (FBS).

8. The cell system according to claim 2, wherein the umbilical mesenchymal stem cells are differentiated by transfecting with an expression system comprising human Nurr1 gene of SEQ ID NO:1.

9. The cell system according to claim 8, wherein the umbilical mesenchymal stem cells are further differentiated by culturing with a composition comprising the murine N-terminal fragment of sonic hedgehog protein and murine FGF8 isoform b in the NCM.

10. The cell system according to claim 1, wherein the neurodegenerative disorders is Parkinson's disease.

11. A cell system for alleviating a syndrome of Parkinson's disease in a mammal, comprising a population of neurons differentiated from human umbilical cord mesenchymal stem cells for expressing tyrosine hydroxylase (TH) in a cell culture.

12. The cell system according to claim 11, wherein the human umbilical cord mesenchymal stem cells are differentiated by culturing with a composition comprising a murine N-terminal fragment of sonic hedgehog protein and a murine FGF8 isoform b in a culture medium.

13. The cell system according to claim 12, wherein the culture medium is an NCM.

14. The cell system according to claim 12, wherein the culture medium is DMEM supplemented with 10% FBS.

15. The cell system according to claim 11, wherein the human umbilical cord mesenchymal stem cells are differentiated by transfecting with an expression system comprising human Nurr1 gene of SEQ ID NO:1.

16. The cell system according to claim 13, wherein the human umbilical cord mesenchymal stem cells are further cultured with a composition comprising the murine N-terminal fragment of sonic hedgehog protein and the murine FGF8 isoform b in the NCM.

17. The cell system according to claim 11, further comprising a population of neurons differentiated from the human umbilical cord mesenchymal stem cells for expressing DBH, GAD, AADC or DAT in the cell culture.

18. A method for treating a neurodegenerative disorder in a mammal, comprising:
differentiating umbilical mesenchymal stem cells into a population of neurons that express at least one of TH, DBH, GAD, AADC and DAT in a cell culture; and
transplanting the population of neurons into the brain of the mammal.

19. The method according to claim 18, wherein the umbilical mesenchymal stem cells are isolated from Wharton's jelly of a human umbilical cord.

20. The method according to claim 19, wherein the umbilical mesenchymal stem cells are differentiated by culturing with a composition comprising a mammalian hedgehog protein and mammalian FGF in a culture medium.

21. The method according to claim 20, wherein the mammalian hedgehog protein includes a murine N-terminal fragment of sonic hedgehog protein.

22. The method according to claim 20, wherein the mammalian FGF includes a murine FGF8 isoform b.

23. The method according to claim 20, wherein the culture medium is an NCM.

24. The method according to claim 20, wherein the culture medium is DMEM supplemented with 10% FBS.

25. The method according to claim 19, wherein the umbilical mesenchymal stem cells are differentiated by transfecting with an expression system comprising human Nurr1 gene of SEQ ID NO:1.

26. The method according to claim 25, wherein the umbilical mesenchymal stem cells are further differentiated by culturing with a composition comprising the murine N-terminal fragment of sonic hedgehog protein and murine FGF8 isoform b in the NCM.

27. The method according to claim 18, wherein the neurodegenerative disorder is Parkinson's disease.

28. The method according to claim 18, wherein the cell culture is transplanted into the brain's striatum of the mammal.

29. The method according to claim 20, wherein the mammal is a human.

30. A method for alleviating symptoms of Parkinson's disease in a mammal, comprising:
isolating human umbilical mesenchymal stem cells from Wharton's jelly of a human umbilical cord;
differentiating the human umbilical mesenchymal stem cells into neurons that express TH in a cell culture; and
transplanting the neurons into the brain of the mammal.

31. The method according to claim 30, wherein the human umbilical cord mesenchymal stem cells are differentiated by culturing with a composition comprising a murine N-terminal fragment of sonic hedgehog protein and a murine FGF8 isoform b in a culture medium.

32. The method according to claim 31, wherein the culture medium is a neuronal conditioned medium.

33. The method according to claim 31, wherein the culture medium is DMEM supplemented with 10% FBS.

34. The method according to claim 30, wherein the human umbilical cord mesenchymal stem cells are differentiated by transfecting with an expression system comprising human Nurr1 gene of SEQ ID NO:1.

35. The method according to claim 34, wherein the human umbilical cord mesenchymal stem cells are further cultured with a composition comprising a murine N-terminal fragment of sonic hedgehog protein and a murine FGF8 isoform b in the NCM.

36. The method according to claim 30, wherein the neurons include neurons that express DBH, GAD, AADC or DAT.

37. The method according to claim 30, wherein the neurons are transplanted into the brain's striatum of the mammal.

38. The method according to claim 30, wherein the mammal is a human.
